SEQUENCE LISTING (11 sequences)

SEQ ID NO: 1 - protein, 5 aa, Artificial Sequence
  Description of Artificial Sequence Synthetic peptide
LSXPP

SEQ ID NO: 2 - protein, 6 aa, Artificial Sequence
  Description of Artificial Sequence Synthetic peptide
LTPAXA

SEQ ID NO: 3 - protein, 7 aa, Artificial Sequence
  Description of Artificial Sequence Synthetic peptide
MTTPPLT

SEQ ID NO: 4 - protein, 5 aa, Artificial Sequence
  Description of Artificial Sequence Synthetic peptide
LTRPX

SEQ ID NO: 5 - protein, 6 aa, Artificial Sequence
  Description of Artificial Sequence Synthetic peptide
MTAXPI

SEQ ID NO: 6 - protein, 4 aa, Artificial Sequence
  Description of Artificial Sequence Synthetic peptide
KKGG

SEQ ID NO: 7 - protein, 11 aa, Artificial Sequence
  Description of Artificial Sequence Synthetic peptide
KKGGFQTPPQL

SEQ ID NO: 8 - protein, 10 aa, Artificial Sequence
  Description of Artificial Sequence Synthetic peptide
RGAVYAENCI

SEQ ID NO: 9 - protein, 11 aa, Artificial Sequence
  Description of Artificial Sequence Synthetic peptide
KGKGQPLFTPQ

SEQ ID NO: 10 - DNA, 1318 bp, Homo sapiens
  CDS (127)..(945)
ggggccgcactagtgattctggttcggcccacctctgaaggttccagaatcgatagtgaattcgtgattttagtttgttgaagttcgtgactgcttcactctctcattcttagcttgaatttggaaatgacttttgatgacctaaagatccagactgtgaaggaccagcctgatgagaagtcaaatggaaaaaaagctaaaggtcttcagtttctttactctccatggtggtgcctggctgctgcgactctaggggtcctttgcctgggattagtagtgaccattatggtgctgggcatgcaattatcccaggtgtctgacctcctaacacaagagcaagcaaacctaactcaccagaaaaagaaactggagggacagatctcagcccggcaacaagcagaagaagcttcacaggagtcagaaaacgaactcaaggaaatgatagaaacccttgctcggaagctgaatgagaaatccaaagagcaaatggaacttcaccaccagaatctgaatctccaagaaacactgaagagagtagcaaattgttcagctccttgtccgcaagactggatctggcatggagaaaactgttacctattttcctcgggctcatttaactgggaaaagagccaagagaagtgcttgtctttggatgccaagttgctgaaaattaatagcacagctgatctggacttcatccagcaagcaatttcctattccagttttccattctggatggggctgtctcggaggaaccccagctacccatggctctgggaggacggttctcctttgatgccccacttatttagagtccgaggcgctgtctcccagacatacccttcaggtacctgtgcatatatacaacgaggagctgtttatgcggaaaactgcattttagctgccttcagtatatgtcagaagaaggcaaacctaagagcacagtgaatttgaaggctctggaagaaaagaaaaaagtctttgagttttattctggaatttaagctattctttgtcacttgggtgccaaacatgagagcccagaaaactgtcatttagctggctgcagaactcctttgcagaaactggggttccaggtgcctggcacctttatgtcaacatttttgattctagctatctgtattatttcacctagcttgtcccaagcttccctgccagcctgaagtccattttcccctttttattttaaaatttgactcctcttcaagcttgaaaaccctctgaactcagtcttctttacctcattatcaccttcccctcacactcctaaaattgcatgaaagacagaccggaattc

SEQ ID NO: 11 - protein, 273 aa, Homo sapiens
MTFDDLKIQTVKDQPDEKSNGKKAKGLQFLYSPWWCLAAATLGVLCLGLVVTIMVLGMQLSQVSDLLTQEQANLTHQKKKLEGQISARQQAEEASQESENELKEMIETLARKLNEKSKEQMELHHQNLNLQETLKRVANCSAPCPQDWIWHGENCYLFSSGSFNWEKSQEKCLSLDAKLLKINSTADLDFIQQAISYSSFPFWMGLSRRNPSYPWLWEDGSPLMPHLFRVRGAVSQTYPSGTCAYIQRGAVYAENCILAAFSICQKKANLRAQ

(19) United States
(12) Patent Application Publication
(10) Pub. No.: US 2005/0089470 A1
(43) Pub. Date: Apr. 28, 2005

(54) Labeled peptides for lectin-like oxidized low-density lipoprotein receptor (LOX-1)

(76) Inventors: Bruce Fletcher Johnson, Scotia, NY (US); Faisal Ahmed Syud, Guilderland, NY (US)

Correspondence Address: Toan Vo;GE Global Research Center, Patent and Legal Operation, One Research Circle, Niskayuna, NY, 12309, US

(21) Appl. No.: 10/691,532

(22) Filed: Oct. 24, 2003

(51) Int. Cl. A61K 51/00; A61K 49/00; C07F 5/00

(52) U.S. Cl. 424/1.11; 424/9.6; 534/11; 534/15; 534/14

(57) ABSTRACT

The present invention relates to compounds labeled with imaging agents that also are capable of binding lectin-like oxidized low-density lipoprotein (LOX-1). The labeled compounds are useful for the diagnosis and monitoring of diseases in which inflammation plays a role, such as various cardiovascular diseases including but not limited to atherosclerosis, vulnerable plaque and coronary artery disease, as well as rheumatoid arthritis.

### BACKGROUND OF THE INVENTION

1. Field of the Invention

The present invention relates in general to peptides labeled with imaging agents that also are capable of binding lectin-like oxidized low-density lipoprotein (LOX-1). The LOX-1 receptor compounds are biomolecules that bind to the LOX-1 receptor, which is over-expressed in atherosclerotic lesions and rheumatoid arthritis. The detectable labels include any detectable label, preferably radionuclides for nuclear scintigraphy or positron emission tomography (PET), paramagnetic metal ions or superparamagnetic particles for magnetic resonance imaging (MRI), heavy metal ions for X-ray or computed tomography (CT), gas-filled microbubbles for targeted ultrasonography (US), or optical dyes for optical imaging, porphyrins or texaphyrins for NMR, fluoresent imaging or photodynamic therapy. The labeled peptides are useful for the diagnosis and monitoring of inflammation and diseases which inflammation plays a role such as various cardiovascular diseases including but not limited to atherosclerosis, vulnerable plaque and coronary artery disease as well as rheumatoid arthritis.

2. Description of Related Art

Atheroschelorosis, a major portion of reported cardiovascular diseases, is the major cause of non-accidental death in Western countries resulting from coronary heart diseases (Coopers, E. S. Circulation, 1993, 24, 629-632; WHO-MONICA Project; Circulation, 1994, 90, 583-612). The progression of the coronary atherosclerotic disease can be divided into five phases (Fuster et al. N. Engl. J. Med. 1992, 326, 242-250), each of which is characterized by a certain level of plaque formation with specific characteristics. Various stages of the general disease progression in and of themselves can be important, such as a vulnerable plaque that can rupture and lead to a blood clot or thrombosis, transient ischemia due to clotting, stroke, myocardial infarction, organ transplant, organ failure and hypercholesterolemia. It is important to detect quantitatively and in a non-invasive manner the progression of the plaque, or a lesion that is in the early stages of plaque formation. Towards this end numerous methods and modalities exit (Vallabhajosula, S. and Fuster, V. J. Nucl. Med. 1997, 38, 1788-1796; Marmion, M. and Deutsch, E. J., Nucl. Biol. Med. 1996, 40, 121-131; Cerqueira, M. D., Seminars Nucl. Med. 1999, 29, 339-351; Narula, J. J., Nucl. Cardiol. 1999, 6, 81-90; Narula, J. Nucl. Med. Commun. 2000, 21, 601-608; Meaney et al., J. Magn. Reson. Imaging 1999, 10, 326-338; Knopp et al., J. Magn. Reson. Imaging 1999, 10, 314-316; Goyen et al., Eur. J. Radiol. 2000, 34, 247-256; Becker et al., Eur. Radiol. 2000, 10, 629-635). However, all these methods provide morphological information rather than chemical or functional information on the plaque or lesion itself, which ultimately is more indicative of disease progression.

Several invasive and noninvasive techniques are routinely used to image atherosclerosis and to assess the progression and stabilization of the disease. These include coronary angiography, intravascular ultrasound angioscopy, intravascular magnetic resonance imaging, and thermal imaging of plaque using infrared catheters. These techniques have been used successfully to identify vulnerable plaques. However, these techniques are generally invasive, requiring surgery, insertion of probes, cameras, or other invasive procedures.

Soluble markers, such as P-selectin, von Willebrand factor, Angiotensin-converting enzyme (C 146), C-reactive protein, D-dimer (Ikeda et al., Am. J. Cardiol., 1990, 65, 1693-1696), and activated circulating inflammatory cells are found in patients with unstable angina pectoris, but it is not yet known whether these substances predict infarction or death (Mazzone et al., Circulation, 1993, 88, 358-363.). It is known, however, that the presence of these substances cannot be used to locate the involved lesion.

Temperature sensing elements contained in catheters have been used for localizing plaque on the theory that inflammatory processes and cell proliferation are exothermic processes. For example, U.S. Pat. No. 4,986,671 discloses a fiber optical probe with a single sensor formed by an elastic lens coated with light reflective and temperature dependent material over which is coated a layer of material that is absorptive of infrared radiation. Such devices are used to determine characteristics of heat or heat transfer within a blood vessel. The devices measure parameters including the pressure, flow, and temperature of the blood in a blood vessel. U.S. Pat. No. 4,752,141 discloses a fiberoptic device for sensing temperature of the arterial wall upon contact. However, discrimination of temperature by contact requires knowing where the catheter is to be placed. These techniques using catheters or devices are invasive, and sometimes may result in or trigger plaque formation or rupture.

An angiogram simply reflects luminal diameter and provides a measure of stenosis with excellent resolution. An angiogram, however, does not image the vessel wall or the various histopathological components. Nevertheless, this technique has become the mainstay of the diagnosis of coronary, carotid, and peripheral artery lesions (Galis et al, Proc. Acad. Sci. USA, 1995, 92, 402-406; Ambrose, J. A. In: Fuster, V. (Ed.); Syndromes of Atherosclerosis: correlations of clinical imaging and pathology; Armonk, N.Y.: Futura Publishing Company, Inc., 1996, 105-122; Kohler, T. R. In: Fuster, V. (Ed.); Syndromes of Atherosclerosis: correlations of clinical imaging and pathology; Armonk, N.Y.: Futura Publishing Company, Inc., 1996, 205-223; Dinsmore, R. E. and Rivitz, S. M. In: Fuster, V. (Ed.), Syndromes of Atherosclerosis: correlations of clinical imaging and pathology. Armonk, N.Y.: Futura Publishing Company, Inc., 1996, 277-289), and is the “gold standard” for anatomic diagnosis despite limited specificity and sensitivity.

An angiogram may be useful for predicting a vulnerable plaque, since low-shear regions opposite flow dividers are more likely to develop atherosclerosis (Ku et al., Atherosclerosis 1985, 5, 292-302). However, most patients who develop acute myocardial infarction or sudden death have not had prior symptoms, much less an angiography (Farb et al., Circulation 1995, 93, 17011709). Certain angiographic data have revealed that a regular plaque profile is a fairly specific, though insensitive, indicator of thrombosis (Kaski et al., Circulation 1995, 92, 2058-2065). Such plaques are likely to progress to complete occlusion, while others are equally likely to progress, but less often reach the point of complete occlusion (Aldeman et al., J. Am. Coll. Cardiol. 1993, 22, 1141-1154). Those that do abruptly progress to occlusion actually account for most myocardial infarctions (Ambrose et al., J. Am. Coll. Cardiol. 1988, 12, 56-62; Little et al., Circulation 1988, 78, 1157-1166). One of the major limitations of angiography is that diffuse atherosclerotic disease may narrow the entire lumen of the artery, and as a result, angiography underestimates the degree of stenosis.

The size of the plaque occlusion is not necessarily determinative. Studies show that most occlusive thrombi are found over a ruptured or ulcerated plaque that is estimated to have produced a stenosis of less than 500 of the vessel diameter. Such stenoses are not likely to cause angina or result in a positive treadmill test. In fact, most patients who die of myocardial infarction do not have three-vessel disease or severe left ventricular dysfunction (Farb et al., Circulation 1995, 93, 1701-1709).

Angioscopy is another technique for the visualization of artery walls, rather than the lumen, and for the characterization of atherosclerotic disease. The angioscopy technique reveals the plaque and surface features not seen by angiography. In addition, it allows the observation of the color (red, white or yellow) of the material in the artery, and is therefore highly sensitive for the detection of thrombus. However, it views only the lesion surface and is not representative of the internal heterogeneity of the plaque. As a routine clinical tool, it may not be practical due to the thickness of the catheter and the invasiveness of this technique. U.S. Pat. No. 5,217,456 and U.S. Pat. No. 5,275,594 disclose the use of light that induces fluorescence in tissues, and of laser energy that stimulates fluorescence in non-calcified tissues. These types of devices differentiate healthy tissue from atherosclerotic plaque, but are not clinically useful for differentiating vulnerable plaque from less dangerous, stable plaque.

High-resolution, real-time B-mode ultrasonography with Doppler flow imaging (Duplex scanning) has merged as one of the best modalities for visualization of carotid arteries (Patel et al., Stroke 1995, 26, 1753-1758). Measurements of wall thickness and quantitative analysis of plaque mass and area can be determined. The echogenicity of the plaque reflects plaque characteristics; echoluscent heterogeneous plaque is associated with both intraplaque hemorrhage and lipids, whereas echodense homogeneous plaque is mostly a fibrous plaque. In addition, the configuration of the plaque (mural versus nodular) can identify active (mural) lesions that are more prone to proliferation and thromboembolism (Weinberger et al., J. Am. Med. Assoc. 1995, 12, 1515-1521). Because the technique is not invasive, it can be used to evaluate the efficacy of drug treatment and to study the natural history of atheroscolerosis (longitudinal studies) by follow-up of individuals at increased risk of atherosclerosis. In coronary and peripheral arteries of low extremities, however, Duplex scanning is clinically not as useful as the traditional angiography.

Atherosclerotic calcification is an organized and regulated process and is found more frequently in advanced lesions, although it may occur in small amount in early lesions (Erbel et al., Eur. Heart J. 2000, 21, 720-732; Wexler et al., Circulation 1996, 94, 11751192). There is a strong association between coronary calcium and obstructive coronary artery disease, and is clearly shown that the amount of coronary calcium was a useful predictor of the extent of coronary artery disease (Agatson et al., J. Am. Coll. Cardiol. 1990, 15, 827-832; Schmermund et al., Am. J. Cardiol. 2000, 86, 127-132; Budoff et al., Am. J. Cardiol. 2000, 86, 8-11). MRI, fluoroscopy, electron beam CT (EBCT), and helical CT can identify calcific deposits in blood vessels. However, only EBCT can quantitate the amount or volume of calcium (Wexler et al., Circulation 1996, 94, 1175-1192). In addition, the EBCT images of the myocardium can be obtained in 0.1 sec. Because of the rapid image acquisition time, motion artifacts are eliminated (Brundage et al. In: Fuster, V. (Ed.). Syndromes of Atherosclerosis: correlations of clinical imaging and pathology. Armonk, N.Y.: Futura Publishing Company, Inc., 1996, 417-427). It has been well-documented that the presence of coronary artery calcium, detected by EBCT, may be a sensitive early marker for the presence and progression of atheroclerotic lesion before the development of complicated lesions (Janowitz et al., Am. J. Cardiol. 1993, 72, 247-254).

One of the limitations with using EBCT for the characterization of calcium in the plaque is reproducibility (Becker et al., Eur. Radiol. 2000, 10, 629-635). In particular the reproducibility of small and very small calcium scores (<100) is lower than that for higher score values. In addition, coronary calcium screening can not reveal atherosclerotic plaque that has little or no calcification-and such soft, lipid-rich plaques are perhaps the most dangerous of all, vulnerable to rupture as a result of hemodynamic stress or inflammation (Carrington, C., Diagnostic imaging, 2000, (April), 48-53; Doherty et al., Am. Heart J. 1999, 137, 806-814).

As red blood cells and platelets gather at the site of the rupture, a blood clot forms and blocks the artery, causing a heart attack. Biologically, calcium may not be the ideal marker because a calcified lesion is presumably a stable lesion, less prone to rupture. More recent data show that coronary calcium scores do not seem to predict myocardial perfusion deficits, plaque burden, or cardiovascular events (Rumberger, J. A. Circulation 1998, 97, 2095-2097; Polak, J. F. Radiology 2000, 216, 323-324).

Magnetic resonance techniques using gradient echo methods to generate images of flowing blood as positive contrast within the lumen of vessels are similar to conventional angiography techniques (Doyle, M. and Pohost, G. In: Fuster, V. (Ed.). Syndromes of Atherosclerosis: correlations of clinical imaging and pathology. Armonk, N.Y.: Futura Publishing Company, Inc., 1996, 313-332; Grist, T. and Turski, P. A. In: Fuster, V. (Ed.). Syndromes of Atherosclerosis: correlations of clinical imaging and pathology. Armonk, N.Y.: Futura Publishing Company, Inc., 1996, 333-362). Magnetic resonance angiography (MRA) of coronary arteries is currently under development, and the resolution is within the range of 1 mm³. MRA techniques provide images of the vessel lumen, whereas MRI studies are often performed to evaluate the effects of the disease on the tissue supplied by the vessel. Recent developments in high-resolution (0.4 mm), fast spin-echo imaging and computer processing techniques visualize in vivo, atherosclerotic plaque activity and intimal thickening (Yuan et al., J. Magn. Reson. Imaging 1994 4, 43-49).

In a recent clinical study in patients with carotid atherosclerosis, MRT was the first non-invasive imaging modality to allow the discrimination of lipid cores, fibrous caps, calcification, normal media, adventia, intraplaque hemorrhage, and acute thrombosis (Toussaint et al., Atheroscler. Thromb. 1995, 15, 15331542; Toussaint et al., Circulation 1996, 94, 932-938). The key advantage of contrast-enhanced rapid imaging techniques is the ability to provide detailed “functional information” with high accuracy (McVein, E. R. Magn. Reson. Imaging 1996, 14, 137-150; Glover, G. D. and Herfkins, R. J. Radiology 1998, 207, 289-235).

In the last two decades, many radiotracers have been developed based on several molecules and cell types involved in atherosclerosis. The potential utility of these radiotracers for imaging atherosclerotic lesions has been studied in animal models, and has been recently reviewed (Vallabhajosula, S. and Fuster, V. J. Nucl. Med. 1997, 38, 1788-1796; Cerqueira, M. D. Seminars Nucl. Med. 1999, 29, 339-351; Narula, J. J. Nucl. Cardiol. 1999, 6, 81-90; Narula, J. Nucl. Med. Commun. 2000, 21, 601-608). In general, radiolabeled proteins and platelets have shown some clinical potential as imaging agents of atherosclerosis, but due to poor target/background and target/blood ratios, these agents are not ideal for imaging coronary or even carotid lesions. Radiolabeled peptides, antibody fragments and metabolic tracers like FDG appear to offer new opportunities for nuclear scintigraphic techniques in the noninvasive imaging of atherothrombosis. However, noninvasive imaging of atherosclerosis remains a challenge for nuclear techniques mainly due to their intrinsic shortcomings, such as low resolution, compared to MRI and CT.

Most of these techniques identify some of the morphological and functional parameters of atherosclerosis and provide qualitative or semiquantitative assessment of the relative risk associated with the disease. Knowledge of the composition of an atherosclerotic plaque may provide a window on the progression of the lesion, which may result in the development of specific therapeutic strategies for intervention. However, these diagnostic procedures are either invasive or yield little information on the underlying pathophysiology such as cellular composition of the plaque, and biological characteristics of each component in the plaque at the molecular level.

The presence of a high amount of oxidized low-density lipoprotein (oxLDL) is noted in the plaque region. It is possible that the lipid pool observed in plaques is fed by the uptaken oxLDL rather than healthy or native low-density lipoprotein (LDL). The oxLDL typically is incorporated into the plaque by the initial recognition of scavenger receptors on macrophages, whereby the macrophages ultimately convert to the foam cells composing the vulnerable plaque (Steinberg, D., J. Biol. Chem. 1997 272 29063-29066). Efforts have been made to track the oxLDL by various means. For example, an antibody against oxLDL radiolabeled with ¹²⁵I has been shown to accumulate in plaques (Tsimikas, S. Arteriosclerosis, Thrombosis, and Vascular Biology 2000, 20, 689-697). In another example, labeled OxLDL has been observed to localize in atherosclerotic plaques (Shaish, A. et al., Pathobiology 2001 69 225-229; Iuliano, L. et al., Atherosclerosis 1996 126 131-141). While other examples exist, none of these methods have resulted in satisfactory early detection of vulnerable atherosclerotic plaque.

It may also be possible to follow the progression of plaques by quantitatively detecting receptors for oxLDL. Recently, a lectin-like oxidized LDL receptor (LOX-1) expressed on endothelial cells has been reported; furthermore, it has been shown to mediate internalization of oxLDL by endothelial cells (Sawamura, T., Nature 1997 386 73-77). Distinct from macrophage scavenger receptors, LOX-1 also is expressed on macrophages and may mediate internalization into cells expressing LOX-1 by first recognizing oxLDL (Yoshida, H., et al. Biochem. J. 1998 334 9-13). LOX-1 is expressed selectively at low levels on endothelial cells, and thereby nearly undetectable in healthy human aorta samples, but is found upregulated in dysfunctional endothelial cells as in atherosclerotic plaque, particular in early lesions that are unlikely to be detectable by other means (Kataoka, H., et al. Circulation 1999 99 3110-3117). It also has been speculated that the upregulation of LOX-1 aids in the production of adhesion receptors that in turn mediate monocyte binding to the cells. Upon penetrating the endothelial wall, the monocytes differentiate into macrophages that may end up as foam cells in the growing plaque.

Detection of LOX-1 in vivo requires ligands that can specifically recognize and strongly bind the receptor while carrying a signal generating entity for a modality such as MRI or PET. Of various classes of ligands demonstrating specificity, such as antibodies, small molecules, or peptides, few examples of identified ligands exist. An antibody to LOX-1 has been developed and used to detect LOX-1 (WO 0164862). The patent claims to be useful in treating atherosclerosis by preventing binding of oxLDL to LOX-1. White, S. et al. have recently reported 60 peptide sequences, generated from phage display, which are described as containing a number of potential consensus motifs that may specifically bind LOX-1 (Hypertension 2001 37, 449-455). The peptides were generated for the development of targeted gene transfer aimed at dysfunctional endothelium. Neither the antibody nor peptides have been considered as potential agents that may be conjugated to MRI or PET agents for the detection of LOX-1, and in turn the progression of cardiovascular diseases.

The description herein of disadvantages and deleterious properties and/or results achieved with known products, methods, and apparatus, is in no way intended to limit the scope of the invention. Indeed, the present invention may utilize one or more known products, methods, and apparatus without suffering from the described disadvantages and deleterious properties and/or results.

### SUMMARY OF THE INVENTION

As described above, a need exists to develop non-invasive methods to diagnose and monitor various cardiovascular diseases (e.g., atherosclerosis, vulnerable plaque, coronary artery disease, renal disease, thrombosis, transient ischemia due to clotting, stroke, myocardial infarction, organ transplant, organ failure and hypercholesterolemia). The non-invasive method should yield information regarding the underlying pathophysiology of the plaque, such as the cellular composition of the plaque and biological characteristics of each component in the plaque at the molecular level.

There also is a need to develop an imaging agent/molecule that is capable of binding LOX-1 and being imaged by external non-invasive imaging techniques. There also is a need to develop a method of making such an imaging agent/molecule, as well as a method of imaging a subject to assess the subject's susceptibility to and to assess the efficacy of therapies for atherosclerosis, vulnerable plaque, coronary artery disease, renal disease, thrombosis, transient ischemia due to clotting, stroke, myocardial infarction, organ transplant, organ failure and hypercholesterolemia. It therefore is a feature of the invention to provide an imaging agent/molecule that is capable of binding LOX-1 in vivo to enable the detection of, and hence, quantitation of the expression of the LOX-1 protein.

In accordance with these and other features of various embodiments of the invention, there is provided a compound having the formula S-(L)ₙ-A, wherein S provides a signal that can be detected in vivo or detected in vitro, L links S to A, A is a peptide comprising an amino acid sequence that specifically binds the LOX-1 receptor, and n is either 0 or 1.

In accordance with another feature of an embodiment of the invention, there is provided a method of making a compound having the formula S-(L)ₙ-A, wherein S provides a signal that can be detected in vivo or detected in vitro, L links S to A, A is a peptide comprising an amino acid sequence that specifically binds the LOX-1 receptor, and n is either 0 or 1, the method comprising synthesizing a peptide A that binds to LOX-1; optionally synthesizing a ligand (L); and attaching a signal-generating component (S) to the peptide A or ligand L.

In accordance with another feature of an embodiment of the invention, there is provided a composition comprising the above-described compound. Another feature of the invention is a method of detecting and quantifying LOX-1 in a mammal comprising administering to a mammal suspected of a disease or disorder caused by expression of LOX-1 the above-described compound, imaging the mammal, and detecting the presence and relative quantity of LOX-1 in the imaged area. The method also includes repeating the above procedure periodically to monitor the quantity of LOX-1 to assess the efficacy of putative therapies for treating diseases or disorders caused by the expression of LOX-1.

These and other features of the invention will be readily apparent to those skilled in the art upon reading the detailed description that follows:

### BRIEF DESCRIPTION OF THE DRAWINGS

FIG. 1 is a transmitted light image overlaid against a fluorescent confocal image of human coronary artery endothelial cells (HCAE) after being contacted with a peptide of the present invention and imaged in accordance with Example 1.

FIG. 2 is the DNA sequence for human LOX-1.

FIG. 3 is a fluorescent image and FIG. 4 is a transmitted light image and of HCAE cells bound to fluorescein-labeled polyclonal antibody for LOX-1 in accordance with Example 2.

FIG. 5 is a transmitted light image overlaid against a fluroescent confocal image of human coronary artery endothelial cells after being contacted with a scrambled peptide in accordance with Example 3.

### DETAILED DESCRIPTION OF PREFERRED EMBODIMENTS

Embodiments of the invention are not limited to the particular methodology, protocols, cell lines, vectors, and reagents described, as these may vary. It also is to be understood that the terminology used herein is for the purpose of describing particular embodiments only, and is not intended to limit the scope of embodiments the present invention which will be limited only by the appended claims.

As used throughout this disclosure, the singular forms “a,” “an,” and “the” include plural reference unless the context clearly dictates otherwise. Thus, for example, a reference to “a host cell” includes a plurality of such host cells, and a reference to “an antibody” is a reference to one or more antibodies and equivalents thereof known to those skilled in the art, and so forth.

Unless defined otherwise, all technical and scientific terms used herein have the same meanings as commonly understood by one of ordinary skill in the art to which embodiments of this invention belongs. Although any methods and materials similar or equivalent to those described herein can be used in the practice or testing of the present invention, the preferred methods, devices, and materials are now described. All publications mentioned herein are cited for the purpose of describing and disclosing the various molecules, amino acid sequences, cell lines, vectors, and methodologies that are reported in the publications and that might be used in connection with the invention. Nothing herein is to be construed as an admission that the invention is not entitled to antedate such disclosures by virtue of prior invention.

Throughout this description, the phrase “S provides a signal that can be detected in vivo or detected in vitro” denotes an entity that can be imaged by itself or by reacting with another substance, and that can be detected in vivo or in vitro by a detection apparatus. More specifically, the labeling entities S include enzymes, fluorescent materials, chemiluminescent materials, biotin, avidin, radioisotopes, radionuclides, X-ray imaging agents, MRI contrast agents, ultrasonography imaging elements, paramagnetic materials, and the like. Suitable imaging agents that provide a detectable signal (S) will be described in more detail below.

The term “fragment” refers to a protein or polypeptide that consists of a continuous subsequence of the subject amino acid sequence and includes naturally occurring fragments such as splice variants and fragments resulting from naturally occurring in vivo protease activity. Such a fragment may be truncated at the amino terminus, the carboxy terminus, and/or internally (such as by natural splicing). Such fragments may be prepared with or without an amino terminal methionine.

The term “variant” refers to a protein or polypeptide in which one or more amino acid substitutions, deletions, and/or insertions are present as compared to the subject amino acid sequence and includes naturally occurring allelic variants or alternative splice variants. The term “variant” includes the replacement of one or more amino acids in a peptide sequence with a similar or homologous amino acid(s) or a dissimilar amino acid(s). There are many scales on which amino acids can be ranked as similar or homologous. (Gunnar von Heijne, Sequence Analysis in Molecular Biology, p. 123-39 (Academic Press, New York, N.Y. 1987.) Preferred variants include alanine substitutions at one or more of amino acid positions. Other preferred substitutions include conservative substitutions that have little or no effect on the overall net charge, polarity, or hydrophobicity of the protein. Conservative substitutions are set forth in Table 1 below.

TABLE 1
Conservative Amino Acid Substitutions

| | |
|---|---|
| Basic: | arginine |
| | lysine |
| | histidine |
| Acidic: | glutamic acid |
| | aspartic acid |
| Uncharged Polar: | glutamine |
| | asparagine |
| | serine |
| | threonine |
| | tyrosine |
| Non-Polar: | phenylalanine |
| | tryptophan |
| | cysteine |
| | glycine |
| | alanine |
| | valine |
| | proline |
| | methionine |
| | leucine |
| | isoleucine |

Table 2 sets out another scheme of amino acid substitution:

TABLE 2

| Original | |
|---|---|
| Residue | Substitutions |
| Ala | gly; ser |
| Arg | lys |
| Asn | gln; his |
| Asp | glu |
| Cys | ser |
| Gln | asn |
| Glu | asp |
| Gly | ala; pro |
| His | asn; gln |
| Ile | leu; val |
| Leu | ile; val |
| Lys | arg; gln; glu |
| Met | leu; tyr; ile |
| Phe | met; leu; tyr |
| Ser | thr |
| Thr | ser |
| Trp | tyr |
| Tyr | trp; phe |
| Val | ile; leu |

Other variants can consist of less conservative amino acid substitutions, such as selecting residues that differ more significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. The substitutions that in general are expected to have a more significant effect on function are those in which (a) glycine and/or proline is substituted by another amino acid or is deleted or inserted; (b) a hydrophilic residue, e.g., seryl or threonyl, is substituted for (or by) a hydrophobic residue, e.g., leucyl, isoleucyl, phenylalanyl, valyl, or alanyl; (c) a cysteine residue is substituted for (or by) any other residue; (d) a residue having an electropositive side chain, e.g., lysyl, arginyl, or histidyl, is substituted for (or by) a residue having an electronegative charge, e.g., glutamyl or aspartyl; or (e) a residue having a bulky side chain, e.g., phenylalanine, is substituted for (or by) one not having such a side chain, e.g., glycine.

Other variants include those designed to either generate a novel glycosylation and/or phosphorylation site(s), or those designed to delete an existing glycosylation and/or phosphorylation site(s). Variants include at least one amino acid substitution at a glycosylation site, a proteolytic cleavage site and/or a cysteine residue. Variants also include peptides with additional amino acid residues before or after the subject amino acid sequence on linker peptides. For example, a cysteine residue may be added at both the amino and carboxy terminals of a subject amino acid sequence in order to allow the cyclisation of the subject amino acid sequence by the formation of a di-sulphide bond. The term “variant” also encompasses polypeptides that have the subject amino acid sequence with at least one and up to 25 or more additional amino acids flanking either the 3′ or 5′ end of the subject amino acid.

The term “derivative” refers to a chemically modified protein or polypeptide that has been chemically modified either by natural processes, such as processing and other post-translational modifications, but also by chemical modification techniques, as for example, by addition of one or more polyethylene glycol molecules, sugars, phosphates, and/or other such molecules, where the molecule or molecules are not naturally attached to wild-type amino acids so derivatized. Derivatives include salts. Such chemical modifications are well described in basic texts and in more detailed monographs, as well as in a voluminous research literature, and they are well known to those of skill in the art. It will be appreciated that the same type of modification may be present in the same or varying degree at several sites in a given protein or polypeptide.

In addition, a given protein or polypeptide may contain many types of modifications. Modifications may take place anywhere in a protein or polypeptide, including the peptide backbone, the amino acid side-chains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, glycosylation, lipid attachment, sulfation, γ-carboxylation of glutamic acid residues, hydroxylation and ADP-ribosylation, selenoylation, sulfation, transfer-RNA mediated addition of amino acids to proteins, such as arginylation, and ubiquitination. PROTEINS—STRUCTURE AND MOLECULAR PROPERTIES, 2nd Ed., T. E. Creighton, W. H. Freeman and Company, New York (1993) and Wold, F., Posttranslational Protein Modifications: Perspectives and Prospects, pgs. 1-12 in Posttranslational Covalent Modification Of Proteins, B. C. Johnson, Ed., Academic Press, New York (1983); Seifter et al., Meth. Enzymol. 182:626-646 (1990) and Rattan et al., Protein Synthesis: Posttranslational Modifications and Aging, Ann. N.Y. Acad. Sci. 663: 48-62 (1992). The term “derivatives” include chemical modifications resulting in the protein or polypeptide becoming branched or cyclic, with or without branching. Cyclic, branched and branched circular proteins or polypeptides may result from post-translational natural processes and may be made by entirely synthetic methods, as well.

The term “homologue” refers to a protein that is at least 60 percent identical in its amino acid sequence of the subject amino acid sequence, as the case may be, as determined by standard methods that are commonly used to compare the similarity in position of the amino acids of two polypeptides. The degree of similarity or identity between two proteins can be readily calculated by known methods, including but not limited to those described in COMPUTATIONAL MOLECULAR BIOLOGY, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M Stockton Press, New York, 1991; and Carillo H. and Lipman, D., SIAM, J. Applied Math., 48: 1073 (1988). Preferred methods to determine identity are designed to provide the largest match between the sequences tested. Methods to determine identity and similarity are codified in publicly available computer programs.

Preferred computer program methods useful in determining the identity and similarity between two sequences include, but are not limited to, the GCG program package (Devereux, J., et al., Nucleic Acids Research, 12(1): 387 (1984)), BLASTP, BLASTN, and FASTA, Atschul, S. F. et al., J. Molec. Biol., 215: 403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol., 215: 403-410 (1990). By way of example, using a computer algorithm such as GAP (Genetic Computer Group, University of Wisconsin, Madison, Wis.), the two proteins or polypeptides for which the percent sequence identity is to be determined are aligned for optimal matching of their respective amino acids (the “matched span”, as determined by the algorithm).

A gap opening penalty (which is calculated as 3×(times) the average diagonal; the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. A standard comparison matrix (see Dayhoff et al. in: Atlas of Protein Sequence and Structure, vol. 5, supp. 3 [1978] for the PAM250 comparison matrix; see Henikoff et al., Proc. Natl. Acad. Sci USA, 89:10915-10919 [1992] for the BLOSUM 62 comparison matrix) also may be used by the algorithm. The percent identity then is calculated by the algorithm. Homologues will typically have one or more amino acid substitutions, deletions, and/or insertions as compared with the comparison subject amino acid, as the case may be.

The term “fusion protein” or “fusion polypeptide” refers to a protein where one or more of the subject amino acid sequences are recombinantly fused or chemically conjugated (including covalently and non-covalently) to a protein such as (but not limited to) an antibody or antibody fragment like an Fab fragment or short chain Fv. The term “fusion protein” or “fusion polypeptide” also refers to multimers (i.e. dimers, trimers, tetramers and higher multimers) of peptides. Such multimers comprise homomeric multimers comprising one subject peptide, heteromeric multimers comprising more than one subject peptide, and heteromeric multimers comprising at least one subject peptide and at least one other protein. Such multimers may be the result of hydrophobic, hyrdrophilic, ionic and/or covalent associations, bonds or links, may be formed by cross-links using linker molecules or may be linked indirectly by, for example, liposome formation. Various fusion proteins known to bind to the human LOX-1 receptor are disclosed in EP 1046652A1, the disclosure of which is incorporated by reference herein in its entirety.

The term “peptide mimetic” or “mimetic” refers to biologically active compounds that mimic the biological activity of a peptide or a protein but are no longer peptidic in chemical nature, that is, they no longer contain any peptide bonds (that is, amide bonds between amino acids). Here, the term peptide mimetic is used in a broader sense to include molecules that are no longer completely peptidic in nature, such as pseudo-peptides, semi-peptides and peptoids. Examples of peptide mimetics in this broader sense (where part of a peptide is replaced by a structure lacking peptide bonds) are described below. Whether completely or partially non-peptide, peptide mimetics according to this invention provide a spatial arrangement of reactive chemical moieties that closely resemble the three-dimensional arrangement of active groups in the subject peptide on which the peptide mimetic is based. As a result of this similar active-site geometry, the peptide mimetic has effects on biological systems that are similar to the biological activity of the subject peptide.

The peptide mimetics of this invention are preferably substantially similar in both three-dimensional shape and biological activity to the subject peptides described herein. Examples of methods of structurally modifying a peptide known in the art to create a peptide mimetic include the inversion of backbone chiral centers leading to D-amino acid residue structures that may, particularly at the N-terminus, lead to enhanced stability for proteolytical degradation without adversely affecting activity. An example is given in the paper “Tritriated D-ala¹-Peptide T Binding”, Smith C. S. et al., Drug Development Res., 15, pp. 371-379 (1988). A second method is altering cyclic structure for stability, such as N to C interchain imides and lactames (Ede et al. in Smith and Rivier (Eds.) “Peptides: Chemistry and Biology”, Escom, Leiden (1991), pp. 268-270). An example of this is given in conformationally restricted thymopentin-like compounds, such as those disclosed in U.S. Pat. No. 4,457,489 (1985), Goldstein, G. et al., the disclosure of which is incorporated by reference herein in its entirety. A third method is to substitute peptide bonds in the subject peptide by pseudopeptide bonds that confer resistance to proteolysis.

A number of pseudopeptide bonds have been described that in general do not affect peptide structure and biological activity. One example of this approach is to substitute retro-inverso pseudopeptide bonds (“Biologically active retroinverso analogues of thymopentin”, Sisto A. et al in Rivier, J. E. and Marshall, G. R. (eds) “Peptides, Chemistry, Structure and Biology”, Escom, Leiden (1990), pp. 722-773) and Dalpozzo, et al. (1993), Int. J. Peptide Protein Res., 41:561-566, incorporated herein by reference). According to this modification, the amino acid sequences of the peptides may be identical to the sequences of the subject amino acid sequence, except that one or more of the peptide bonds are replaced by a retro-inverso pseudopeptide bond. Preferably the most N-terminal peptide bond is substituted, since such a substitution will confer resistance to proteolysis by exopeptidases acting on the N-terminus. Further modifications also can be made by replacing chemical groups of the amino acids with other chemical groups of similar structure. Another suitable pseudopeptide bond that is known to enhance stability to enzymatic cleavage with no or little loss of biological activity is the reduced isostere pseudopeptide bond (Couder, et al. (1993), Int. J. Peptide Protein Res., 41:181-184, incorporated herein by reference in its entirety).

Thus, the amino acid sequences of these peptides may be identical to the sequences of the subject amino acid sequence, except that one or more of the peptide bonds are replaced by an isostere pseudopeptide bond. Preferably the most N-terminal peptide bond is substituted, since such a substitution would confer resistance to proteolysis by exopeptidases acting on the N-terminus. The synthesis of peptides with one or more reduced isostere pseudopeptide bonds is known in the art (Couder, et al. (1993), cited above). Other examples include the introduction of ketomethylene or methylsulfide bonds to replace peptide bonds.

Peptoid derivatives represent another class of peptide mimetics that retain the important structural determinants for biological activity, yet eliminate the peptide bonds, thereby conferring resistance to proteolysis (Simon, et al., 1992, Proc. Natl. Acad. Sci. USA, 89:9367-9371, incorporated herein by reference in its entirety). Peptoids are oligomers of N-substituted glycines. A number of N-alkyl groups have been described, each corresponding to the side chain of a natural amino acid (Simon, et al. (1992), cited above). Some or all of the amino acids of the subject molecules may be replaced with the N-substituted glycine corresponding to the replaced amino acid.

The term “peptide mimetic” or “mimetic” also includes reverse-D peptides and enantiomers. The term “reverse-D peptide” refers to a biologically active protein or peptide consisting of D-amino acids arranged in a reverse order as compared to the L-amino acid sequence of the subject peptide. The term “enantiomer” refers to a biologically active protein or peptide where one or more the L-amino acid residues in the amino acid sequence of a subject peptide is replaced with the corresponding D-amino acid residue(s).

A “composition” as used herein, refers broadly to any composition containing a described molecule, peptide, or amino acid sequence. The composition may comprise a dry formulation, an aqueous solution, or a sterile composition. Compositions comprising the molecules described herein may be stored in freeze-dried form and may be associated with a stabilizing agent such as a carbohydrate. In use, the composition may be deployed in an aqueous solution containing salts, e.g., NaCl, detergents, e.g., sodium dodecyl sulfate (SDS), and other components, e.g., Denhardt's solution, dry milk, salmon sperm DNA, etc.

Monoclonal antibodies, composed of naturally occurring amino acids, and their fragments have been studied extensively for their potential applications in both diagnostic and therapeutics. For example, Anderson et al. have recently targeted an MRI contrast agent to the αᵥb₃ integrin using antibody technology to enhance the contrast of angiogenic vessels in vivo (Anderson et al., Mag. Res. Med. 2000, 44, 433). Though antibodies have a high receptor binding affinity and high specificity, they often demonstrate limited accumulation in the target and relatively slow blood clearance due to their high molecular weight, resulting in only modest target to-background ratios. Peptides, as in proteins, are compounds that also contain amino acids (R-amino carboxylic acids) linked by amide (peptide) bonds. Designed by nature for stimulating, inhibiting, or regulating numerous life functions, peptides have been considered by many to be ideal agents for therapeutic applications. While proteins also are comprised of natural amino acids, the difference between proteins and peptides is their sizes. As used herein, the term “peptide” denotes amino acid sequences containing less than 100 amino acids with a molecular weight of about 10,000 Da. The expression “small peptides” denotes peptides with less than 30 amino acids or a molecular weight less than 3500 Da. As used throughout this description, the expressions “peptide” and “small peptide” denote peptides, consensus amino acid sequences of the peptide, mimetics, variants, derivatives, and homologues thereof.

Compared to antibodies or proteins, small peptides offer several advantages. Most importantly, affinity can often be modulated by the peptide sequence and structure; coupled with the fact that peptides are relatively easy to synthesize, they offer an advantage in discovery and synthesis over other forms of ligands. In addition, peptides often can tolerate harsher chemical conditions for modification (McAfee, J. G.; Neumann, R. D., Nucl. Med. Biol. 1996, 23, 673) or conjugation to a signal-generating moiety. Moreover, the synthesis of a peptide conjugated to a signal-generating moiety as a whole is significantly simpler and more controlled as described in more detail herein. Small peptides are less likely to be immunogenic. They also are necessary elements in more fundamental biological processes than any other class of molecule, and in many cases the affinities of small peptides for their receptors are significantly greater than that of antibodies or their fragments. Finally, other small molecule targeting agents such as peptidomimetics, or mimetics of the peptide may be inspired by peptide sequences. Many biologically active peptides are known, and some of them are available commercially, thus providing a suitable starting point. Commercially available Phage display technology can also allow one to screen very large libraries of peptides against given targets.

Precedence exists for using peptides as targeting agents for the purpose of imaging as described in more detail herein. Examples include 99mTc labeled chemotactic peptides (Baidoo et al., J. Nucl. Med. 1994, 35, 19P (abstract 67); Babich et al., J. Nucl. Med. 1993, 34, 1964.) leukotriene B4 LTB4 receptor antagonists (Rajopadhye et al., Abstracts of Papers, 216th ACS National Meeting, Boston, Mass., 1998; American Chemical Society: Washington, D.C., 1998; abstract 005 (medicinal chemistry); Barrett et al., J. Nucl. Med. 1998, 39, 215P (abstract 957), and tuftsin receptor antagonists (Goodbody et al., Eur. J. Nucl. Med. 1994, 21, 790 (abstract 262)) for imaging focal sites of infection; somatostatin analogues (Maina et al., Eur. J. Nucl. Med. 1994, 21, 437.), bombesin analogues (Baidoo et al., Bioconjugate Chem. 1998, 9, 218.), folate receptor antagonists (Wang et al., Bioconjugate Chem. 1997, 8, 673.), and vasoactive intestinal peptide (VIP; Kurtaran et al. J. Nucl. Med. 1997, 38, 880.) for imaging tumors; and platelet GPIIb/IIIa receptor antagonists for imaging thrombi (Barrettet et al., J. Nucl. Med. 1995, 36, 16P (abstract 55); Liu et al., Bioconjugate Chem. 1996, 7, 63; Muto et al. J. Nucl. Med. 1995, 36, 1384.).

An embodiment of the present invention relates to molecules useful in detecting or imaging atherosclerotic tissue by binding to LOX-1. These molecules preferably have the following characteristics:
contain a peptide comprising an amino acid sequence that binds to LOX-1 in the presence of human fluids with adequate specificity such that atherosclerotic tissue may be differentiated from healthy tissue; and
contain a signal moiety that can be detected.

LOX-1 or lectin-like oxidized LDL receptor was recently identified as a receptor on endothelial cells for oxLDL; it mediates the internalization of oxLDL by endothelial cells and is distinct from macrophage scavenger receptors such as those described in WO 2002/06771, (Sawamura, T. Nature 1997 386:73-77). LOX-1 also is expressed on macrophages and may play a role in oxLDL recognition/internalization on these cells (Yoshida, H. et al., Biochem. J. 1998 334:9-13). LOX-1 is nearly undetectable in healthy human aorta samples but is found in atherosclerotic plaque, particular early lesions that are unlikely to be detectable by other means (Kataoka, H. et al., Circulation 1999 99:3110-3117). An antibody to LOX-1 has been described as being useful to treat atherosclerosis by preventing binding of oxLDL to LOX-1 (WO0164862). Recent work suggests that recognition of oxLDL by LOX-1 is a critical early step in expression of adhesion receptors on endothelial cells. These receptors are believed to be responsible for attracting monocytes to the early atherosclerotic plaque. The monocytes penetrate the endothelial, differentiate into macrophages and can end up as foam cells in the growing plaque. Finally, peptides were developed that bind to LOX-1 using phage display technology (White, S. et al., Hypertension 2001 37:449-455).

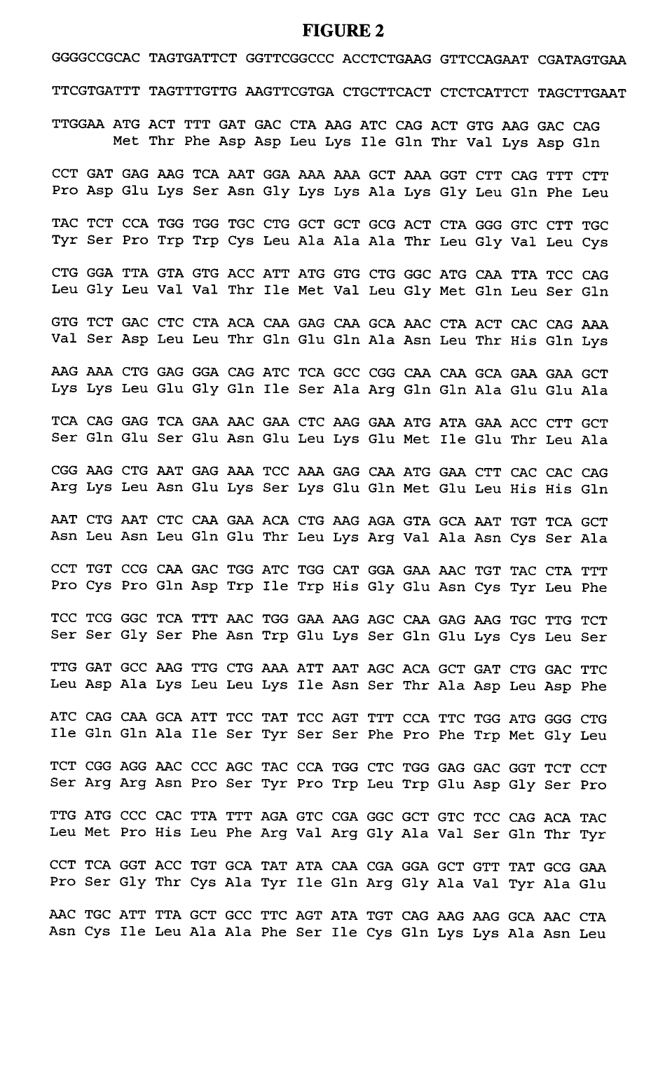

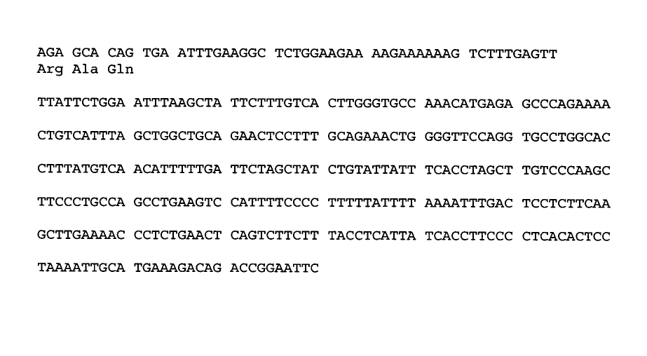

Various species of LOX-1 have been isolated and sequenced revealing relatively significant dissimilarity interspecies (Chen, M., et al., J. Biochem., 355:289-95 (2001). U.S. Pat. Nos. 5,962,260 and 6,197,937, the disclosures of which are incorporated by reference herein in their entirety, disclose the amino acid sequence of human and bovine LOX-1. The DNA sequence of the LOX-1 receptor is shown in FIG. 2. Using the techniques disclosed in these documents, and the guidelines provided herein, those skilled in the art are capable of isolating LOX-1 from any species, and developing and synthesizing peptides comprising amino acid sequences that bind to the human LOX-1.

Thus, in the above molecule, (S-(L)ₙ-A), the moiety that binds to LOX-1 (A) can be synthesized using known techniques, given the known amino acid sequence of the LOX-1 polypeptide. Moieties that bind only specific portions of LOX-1 also can be synthesized given the known and/or expected antigenic determinant or epitope binding site. Unlike known moieties that bind to LOX-1, the inventive moieties are designed to bind to LOX-1 in the presence of human fluids (in vivo or in vitro) with sufficient specificity such that tissue in which LOX-1 has been overexpressed (e.g., atherosclerotic tissue) may be differentiated from healthy tissue. The inventive molecules also are bound, again in the presence of human fluids, to a signal moiety with sufficient specificity to enable detection using imaging techniques.

Moieties that bind to LOX-1 include, for example, antibodies to LOX-1 such as those described in WO 0164862, and U.S. Pat. No. 6,197,937, macromolecules other than antibodies such as poly I and carrageenan (Arterioscler Thromb Vasc Biol. 1998 18:1541-1547.), peptides such as those described in White, S. et al. Hypertension 2001 37 449-455, peptide mimetics and organic molecules that satisfy the criteria above. It is specifically preferred to use in the present invention peptides containing amino acid sequences that specifically bind to the LOX-1 receptor.

The methods disclosed in the above documents can be used to generate a plurality of agents capable of binding LOX-1. These agents then can be screened as described herein by reacting them with a plurality of signal moieties and optional linking ligands, and tested to assess their efficacy in binding both the LOX-1 polypeptide and the signal moieties. Particularly preferred binding moieties A include peptides selected from the group consisting of LSXPP (X=I, R), TPP, LTPAXA (X=T, R), MTTPPLT, LTRPX (X=Y, L), MTAXPI (X=P, R), MQP, and mixtures, fragments, fusion peptides, derivatives, variants, and homologues thereof.

A number of methods can be used to screen and evaluate the binding affinity of different ligands. One method for example includes fluorescent based in vitro experiments. Cell-based assays can simultaneously yield information on the amount of signal generating entity necessary for detection, and therefore required for conjugation to ligands.

For the peptidic ligands of embodiments of the invention, a fluorescent dye preferably is attached to the N-terminus of the peptide via a flexible linker, such as the amino acid sequence KKGG (K=Lysine, G=Glycine). In the event that the N-terminus is linked to a signaling moiety with no further functional ends for dye attachment, the dye also can be attached via the side-chain amine of a K residue incorporated into the sequence (e.g. in the linker).

Regardless of the type of screening assay used, (e.g., a generic in vitro model), it first is assumed that the amount of LOX-1 on the surface of a substrate is known, whether it be cells or some other substrate. In a multi-well transparent plate, LOX-1 is present (either as pure LOX-1 protein or expressed on cells) uniformly across the wells. Labeled ligands then can be added and incubated for an optimized amount of time in the different wells. The wells then are washed thoroughly with a buffer, such as Phosphate buffered saline (PBS), and the plate then imaged while shining a laser to excite and initiate fluorescence of the dye attached to the ligands. The fluorecent intensity from each well, and thus the degree of different ligand binding, can be quantified. To obtain the absolute number of ligand bound, the signal preferably is further calibrated by obtaining the fluorescent intensity of a known quantity of dye-conjugated ligand under similar conditions of the binding assay. If the number of LOX-1 molecule is known, and the amount of bound ligands determined, dissociation constants to evaluate ligand-binding affinity can be calculated. Different ligands can thus be screened quantitatively for their binding affinity. The number of bound ligands per cells also is capable of providing information on parameters required to obtain a detectable signal from a signal-generating entity conjugated to the ligands.

Images in the assay may be acquired using a laser confocal microscope or an Imager. For example, images of peptides bound to cells can be obtained using a laser confocal microscope as follows: HCAE cells can be grown on high quality borosilicate 8-chambered glass slides (Electron Microscopy Sciences, Fort Washington, Pa.). Then, about 10 μL of 1 mg/ml of an aqueous solution of a labeled peptide can be added to the cells and incubated for 1 hour. Subsequently, the cells preferably are washed with HBSS buffer three times. The cells then can be fixed with 4% formaldehyde solution over 10 minutes. After a final wash with buffer, the slide is imaged. Images preferably are acquired using an OLYMPUS laser scanning confocal microscope, model Fluoview 300, using Ar-ion laser (selecting 488 nm line) and a 510-nm long-pass filter. Images can be acquired using two channels: reflected light and fluorescent mode channel, or an overlay of both channels.

For higher throughput screening the method described above can be extended: a 96-well plate may replace the 8-well slides and a Biorad Imager, model FX Proplus, may replace the confocal microscope. For example, images of fluorescein-labeled peptide (or antibody—polyclonal or monoclonal) bound to cells can be obtained using an Imager, whereby HCAE cells can be laid on and grown in wells on a standard commercial 96-well plate (Becton-Dickenson, Franklin Lakes, N.J.). Then, about 10 μL of 1 mg/ml labeled peptide (or antibody—polyclonal or monoclonal) solution can be added to the cells and incubated for 1 hour. Subsequently, the cells preferably are washed with PBS buffer three times. After a final wash with buffer, the slide can be imaged using the Biorad imager selecting “Fluorescein” as the fluorophore.

Linear peptides containing one or more of the above described amino acid sequences may be synthesized using standard automated synthesis protocols using solid-phase peptide synthesis. One example of a solid-phase peptide synthesis is outlined below in reaction scheme 1. These consensus sequences may be further optimized or extended to improve affinity and other in vivo characteristics (e.g. degradability, immunogenicity) via combinatorial chemistry or molecular modeling efforts. Using the guidelines provided herein, and armed with the peptide amino acid sequences described herein, those skilled in the art are capable of synthesizing the peptides, and improving affinity without undue experimentation.

Conjugation to a signal-generating moiety may be achieved via the peptide backbone or side chain using similar peptide chemistry, such as amide bond formation. An example of conjugating the synthesized peptide to a signal-generating moiety is shown below in reaction scheme 2. In a similar vein, cyclic peptides may be generated whereby the linear sequences are cyclized via either backbone bonds or side-chain bonds, as shown in more detail in reaction scheme 3 below.

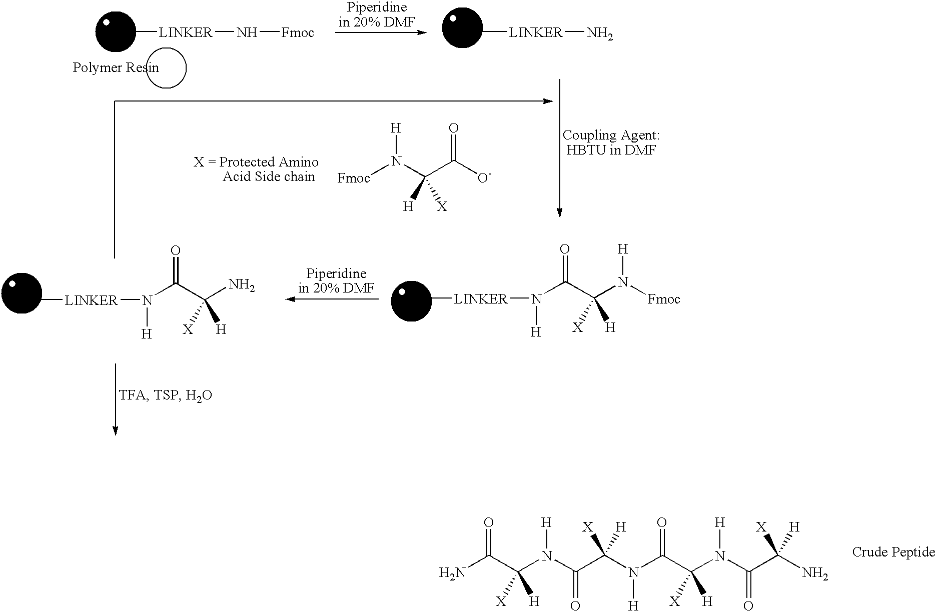

Reaction scheme 1: Automated peptide synthesis. The peptide is synthesized one amino acid at a time on an insoluble polymer resin and subsequently cleaved off from the resin and purified. The abbreviation Fmoc in the reaction scheme refers to fluorenylmethoxycarbonyl.

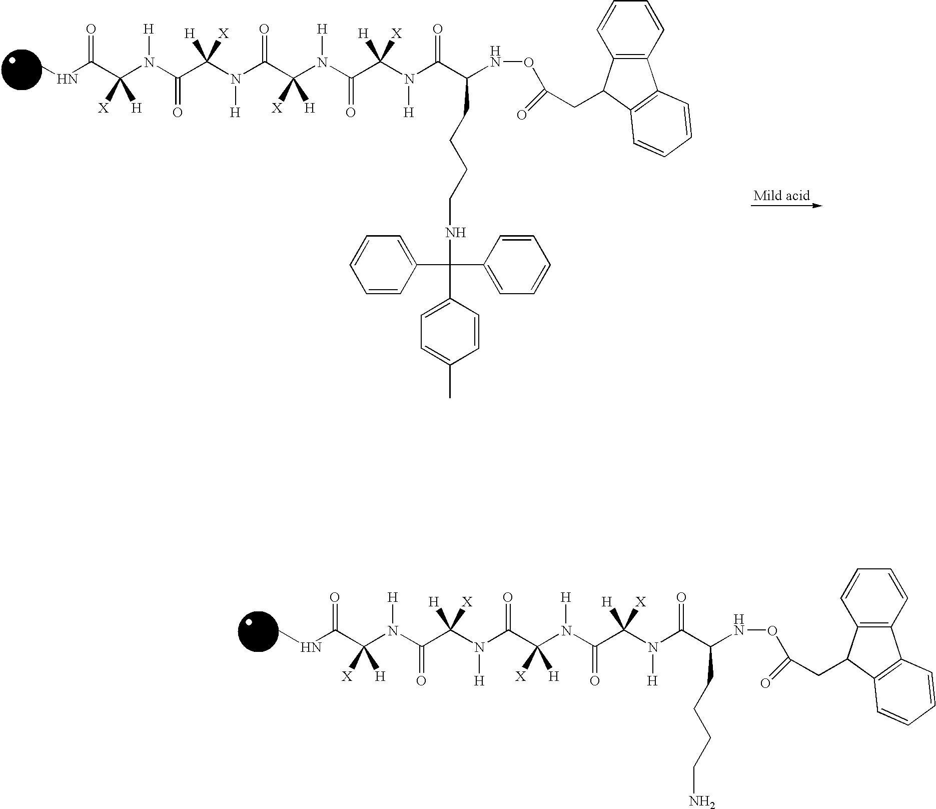

Reaction scheme 2: Side-chain functionalization of a peptide. A peptide may be synthesized on a resin by standard method such that all reactive side-chains are protected. Once a side-chain is deprotected, keeping the backbone protected from any reaction, a moiety may be selectively attached to the side chain. Alternatively, a moiety may be attached to the backbone as in an amino acid. In this regard, the deprotected side-chain containing a free amine group may be reacted using conventional chemistry synthesis with a signal generating moiety, or precursor or conjugate thereof (e.g., conjugate of S-(L)ₙ), to synthesize the compound of the invention.

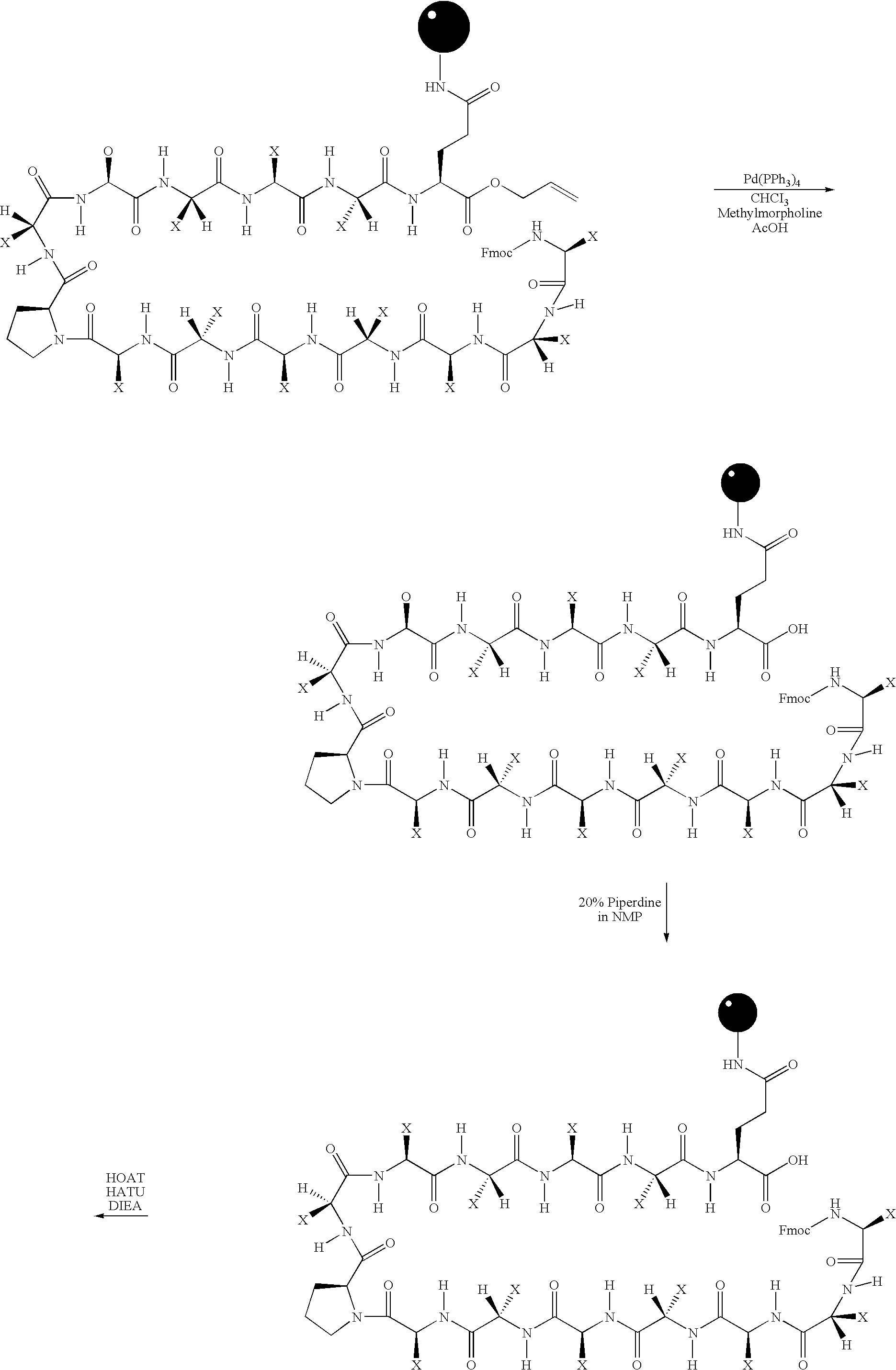

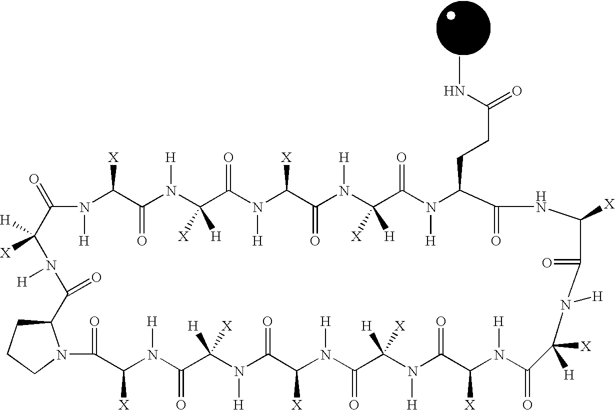

Reaction scheme 3a: Peptide cyclization via backbone. The side-chain of an amino acid is attached to the resin and the peptide lengthened through the backbone by standard peptide chemistry. The peptide is cyclized while still on polymer resin. The letter X in the above reaction scheme is a protected amino acid side chain (e.g., same as letter R in reaction scheme 1).

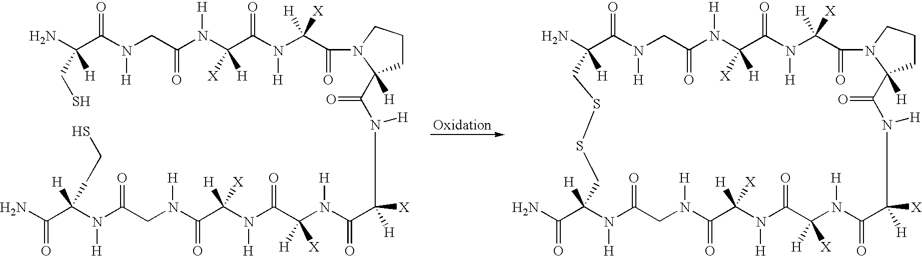

Reaction scheme 3b: Peptide cyclization via side-chain. A purified peptide containing 2 cysteines is used to cyclize the peptide by forming a disulfide bond. The cyclized peptides prepared in reaction schemes 3a or 3b can be conjugated to a signal generating moiety using the techniques disclosed in reaction scheme 2.

The preferred peptide syntheses outlined in the above reaction schemes can be prepared in accordance with the following description. Peptides can be synthesized using standard solid phase techniques with N^{α}-Fmoc-protected amino acids (Sheppard, R. C., Peptides, North-Holland Publishing Company, Amsterdam (1973)) using 2,4-dimethoxybenzhydrylamine resin (Rink Amide AM) on a 25 μmole scale (Fmoc=Fluorenylmethoxycarbonyl). The peptides can be synthesized using a Rainin/Protein Technology Symphony solid phase peptide synthesizer (Woburn, Mass.). Prior to any reaction chemistry, the resin preferably is swelled for one hour in methylene chloride, and subsequently exchanged out with DMF (dimethylformamide) over half-hour or more.

Each coupling reaction to synthesize the peptide can be carried out at room temperature in DMF with five equivalents of amino acid. Reaction times usually are from about 20 minutes to about 3 hours, more preferably about 45 minutes, 1 hour for residues that were expected to be difficult to couple (for example, coupling Isoleucine, I, to proline, P, in the IPP sequence). The preferred coupling reagent used is HBTU (O-Benzotriazolyl-1-yl-N,N′,N′-tetramethyluronium hexafluorophosphate), with NMM (N-methylmorpholine) as the base. During each coupling step, the coupling agent preferably is delivered at a scale of five equivalents relative to the estimated resin capacity, and reaction carried out in 2.5 ml of 0.4 M NMM solution in DMF.

It is preferred that the coupling reactions do not perturb the side-chains of the amino acids because the acids typically were protected with acid labile groups if reactive groups were present. For example, tyrosine, threonine and serine side chains can be protected as the corresponding tert-butyl ethers. Glutamic acid side chains can be protected as the corresponding tert-butyl ester. Lysine and omithine side chains can be Boc protected. Glutamine side chains also may be protected as the γ-triphenylmethyl derivative, and the arginine side chain protected as the 2,2,5,7,8-Pentamethyl-chromane-6-sulfonyl derivative.

Following each coupling reaction, the N-terminal Fmoc-protected amine preferably is deprotected by applying 20% piperidine in DMF twice at room temperature for approximately 15 minutes. After the addition of the last residue the resin, still on the peptide synthesizer, preferably is rinsed thoroughly with DMF and methylene chloride. The signaling moiety (S) can be attached to the peptide for ultimate attachment to the binding moiety (B), or directly to B.

For example, to couple the fluorescein dye, 5(6)-carboxyfluorescein, to the N-terminus of a synthesized peptide, the dye, HBTU and NMM preferably are added to the resin in the same manner as the amino acids described above. After the reaction, the resin preferably is thoroughly washed with DMF and methylene chloride and dried under a stream of nitrogen. A mixture containing 1 mL TFA, 2.5% TSP (triisopropylsilane) and 2.5% water can be used to cleave the peptides from the resin. The resin and mixture preferably are stirred at room temperature for approximately 3 to 4 hours. The resin beads then can be filtered off using glass wool, followed by rinsing with 2-3 ml of TFA. The peptide then preferably is precipitated with ice-cold ether (40 mL) and centrifuged (e.g., at 3000-4000 rpm) until the precipitate formed a pellet at the bottom of the centrifuge tube. The ether can be decanted, and the pellet resuspended in cold ether (40 mL) and centrifuged again—the process can be repeated two to three times. During the final wash 10 ml of Millipore water preferably is added to 30 ml of cold ether, and the mixture was centrifuged again. The ether then can be decanted, the aqueous layer containing the crude peptide then can be transferred to a round bottom flask for lyophilization. Crude yields for peptide synthesis were usually approximately 90%. No unlabeled peptide was typically observed.

Peptides preferably are purified by reverse phase semipreparative or preparative HPLC with a C4-silica column (Vydac, Hesperia, Calif.). The peptide chromatograms can be monitored at 220 nm, which corresponds to the absorption of the amide chromophore. Monitoring at 495 nm also can be observed to ensure the presence of the fluorescein dye on the peptide. It is preferred to use a solvent system including CH₃CN/TFA (acetonitrile/Trifluoroacetic acid; 100:0.01) and H₂O/TFA (water/Trifluoroacetic acid; 100:0.01) eluents at flow rates of 3 ml/min and 10 ml/min for semipreparative and preparative, respectively. Dissolved crude peptides in Millipore water can be injected at a scale of 1.5 mg and 5-10 mg peptide for semipreparative or preparative, respectively. The chromatogram shape was analyzed to ensure good resolution and peak shape. Gradient conditions for all peptides were typically 5 to 50% of CH₃CN/TFA (100:0.01) in 30 minutes. Purified peptide identity was confirmed by matrix-assisted laser desorption time-of-flight mass spectroscopy.

Using the guidelines provided herein and the description immediately above, a person having ordinary skill in the art is capable of synthesizing any group A, which is a peptide comprising an amino acid sequence that binds to LOX-1, and more preferably is selected from the group consisting of LSXPP (X=I, R), TPP, LTPAXA (X=T, R), MTTPPLT, LTRPX (X=Y, L), MTAXPI (X=P, R), MQP, and mixtures, fragments, fusion peptides, derivatives, variants, and homologues thereof. Upon synthesizing the group A peptide, the peptide then is conjugated either directly to a signal moiety, or to the signal moiety via a linking group.

Any signal moiety can be used so long as it is capable of binding the peptide (component A) and generating a detectable signal. Suitable signal moieties include a luminescent dye, a radionuclide, a near infrared dye, a magnetically active isotope, a superparamagnetic particle, a metal ion having a Z value of greater than 50, an encapsulated species, and a combination thereof. The signal moiety may include, for example:
dyes, fluorescent dye, chemiluminescent dyes for optical imaging, histology;
molecules containing high-Z elements, such as iodine, for X-ray imaging, computed tomography (CT);
gas-filled microbubbles, fluorocarbon filled micelles for ultrasonography (US);
paramagnetic ions, such as chelated Gd⁺⁺⁺, or superparamagnetic particles such as superparamagnetic iron oxide nanoparticles (SPIO) for magnetic resonance imaging (MRI); or
radionuclides such as 99mTc or ¹⁸F for Single Photon Emission Computed Tomography (SPECT) or positron emission tomography (PET).

Particularly preferred signal moieties include fluorescein, rhodamine, Alexa, ¹¹C, ¹⁸F, ⁵²Fe ⁶²Cu, ⁶⁴Cu, ⁶⁷Cu, ⁶⁷Ga, ⁶⁸Ga, ⁸⁶Y, ⁸⁹Zr, ^{94m}Tc, ⁹⁴Tc, ^{99m}Tc, ¹¹¹In, ¹²³I, ¹²⁴I, ¹²⁵I, ¹³¹I, ¹⁵⁴⁻¹⁵⁸Gd and ¹⁷⁵Lu, superparamagnetic iron oxide nanoparticles, heavy metal ions, gas-filled microbubbles, optical dyes, porphyrins, texaphyrins, highly iodinated organic compounds chelates thereof, polymers containing at least one of the aforementioned components, endoedral fullerenes containing at least one of the aformentioned, near infrared dyes, Cye dyes, and mixtures thereof. Even more preferably, the signal moieties are F-18 for PET, superparamgnetic iron oxide nanoparticles (SPIO) for MRI, chelated Gd, I, and Y. Most preferably, the signal moiety is F-18 for PET.

¹⁸F-Fluoride can be obtained from cyclotrons after bombardment of ¹⁸-enriched water with protons. Typically, the enriched water containing ¹⁸F-fluoride is treated with a base having a counter-ion that is any alkali metal cation (M⁺), such as potassium or another monovalent ion as well as a chelate for M⁺, such as Kryptofix 222. The water can be evaporated off to produce a residue of chelate M-¹⁸F, which can be taken up in an organic solvent for further use. The purpose of the chelate is to solubilize the M-¹⁸F in the organic solvent and confer nucleophilicity to the ¹⁸F-fluoride. Instead of a chelate and M⁺, a quaternary ammonium salt, phosphonium salt or guandinium may be used to solubilize the ¹⁸F-fluoride in the organic solvent and confer nucleophilic reactivity to the ¹⁸F-fluoride. Potassium is generally used as a counter-ion.

Because fluoride is the most electronegative element, it has a tendency to become hydrated and lose its nucleophilic character. To minimize this, the labeling reaction preferably is performed under anhydrous conditions. For example, fluoride (as potassium fluoride or as a complex with any of the other counter-ions discussed above) can be placed in organic solvents, such as acetonitrile or THF. With the assistance of agents that bind to the counter-ion, such as Kryptofix 2.2.2(4,7,13,16,21,24-hexaoxa-1,10-diazabicyclo[8.8.8]-hexacosane), the fluoride ion is very nucleophilic in these solvents. The remaining portion of the chelate molecule of the invention then can be added to the solvent and the chelate thereby labeled with the F-¹⁸. Using the guidelines provided herein, those skilled in the art are capable of labeling the ligands of embodiments of the present invention with F-¹⁸. Alternatively, labeling may be accomplished through the use of ¹⁸F-F₂ or electrophilic fluorinating agents derived from ¹⁸F-F₂.

Other suitable signaling moieties include magnetically active isotopes, such a paramagnetic ions, isotopes of gadolinium, and polymers containing such compounds. Nanoparticles of iron oxides or elemental iron also can be used as superparamagnetic signaling agents. Components having a Z value greater than about 50, such as iodine and bismuth also can be used. Suitable signal moieties further include encapsulated species such as micelles, liposomes, polysomes, and gas-filled microbubles.

L is simply any moiety which connects the signal moiety S to the peptidic binding moiety A. In the case of ¹⁸F or 11C a linker may not be necessary; the radioisotope can be directly attached to A via a covalent bond. In many cases it is preferred to include L in order to attach S to A. That is, n in the equation for the molecule of the invention is 1. Preferred linking agents include polypeptides, proteins, and small organic moieties. For example, lysine-glycine analogs, derivatives and variants can be used, conventional chelators such as cyclohexyl alanine, DTPA, 1,4,7-triaza-cyclononane-N,N′,N″-triacetic acid (NOTA), p-bromoacetamido-benyl-tetraethylaminetetraacetic acid (TETA), 1,4,7,10-tetraazacyclododecanetetraacetic acid (DOTA), and combinations thereof. A preferred linking agent could be a lysine-glycine derivative such as KKGG

Organic moieties having a valence of at least 2 are useful as L in the above formula, including small organic moieties such as benzoate or propionate. The organic radical may be covalently bound to both S and A, or it may be ionically bound to S, A, or both S and A. The organic moiety suitable for use as the linking agent typically has from aout 1 to about 10,000 carbon atoms, and may include, an organic radical selected from the group consisting of alkylene, arylene, cycloalkylene, aminoalklylene, aminoarylene, aminocycloalkylene, thioalkylene, thioarylene, thiocycloalkylene, oxyalkylene, oxyarylene, oxycycloalkylene, acylalkylene, acylarylene, acylcycloalkylene units, and combinations thereof. A particularly preferred acylarylene unit is a 4-acylphenylene group having the structure below:

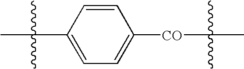

Other suitable linking agents including metal chelating agents, such as one or more of DTPA, 1,4,7-triaza-cyclononane-N,N′,N″-triacetic acid (NOTA), p-bromoacetamido-benyl-tetraethylaminetetraacetic acid (TETA), 1,4,7,10-tetraazacyclododecanetetraacetic acid (DOTA), EDTA, and CHXa. It is preferred that the metal chelating agents be capable of binding to at least one metal selected from cations of ⁵²Fe, ⁶²Cu, ⁶⁴Cu, ⁶⁷Cu, ⁶⁷ Ga, ⁶⁸Ga, ⁸⁶Y, ⁸⁹Zr, ^{94m}Tc, 94Tc, ^{99m}Tc, ¹¹¹In, ¹⁵⁴⁻¹⁵⁸Gd, and ¹⁷⁵Lu.

As appreciated by those skilled in the art, various linking agents are used with certain signal moieties. For example, signal generating moieties, such as ⁶⁴Cu, typically require a linking ligand, whereas ¹⁸F does not. In addition, labeled prosthetic groups such as ¹⁸F-fluoropropionate or ¹⁸F-fluorobenzoate can be used such that, once prepared, they can be conjugated to the peptide via active ester conjugation. Those skilled in the art are capable of synthesizing a suitable linking agent, if needed, together with a suitable signaling moiety, using the guidelines and synthesis techniques provided herein.

The labeled compounds of the present invention can be used as a diagnostic to assist in imaging a targeted tissue that is suspected of overexpressing LOX-1. The method of diagnosis therefore includes first administering to a subject a composition containing the labeled compound of the invention. The method also optionally includes administering a clearing agent to assist in clearing any unbound binding compound or fragments thereof from circulation. Depending on the particular label that has been labeled to the ligand, the appropriate imaging technique is employed to image the targeted tissue. For example, when F-18 is used as the labeling agent PET imaging is conducted and the targeted tissue, which now is bound to the compounds of the invention, can be seen.

The imaging method can be used as a diagnostic to detect the presence of LOX-1 in human tissue. In addition, the imaging method can be repeated over a number of days to provide a quantitative assessment of the degree of growth or expression of the LOX-1 polypeptide. Repeating the imaging method also provides for an assessment of the efficacy of putative therapies for diseases and disorders caused by the overexpression of LOX-1.

In addition, the labeled compounds of the invention can be used in a pretargeting regimen where a bispecific antibody first is genetically engineered using techniques known in the art, whereby one arm of the bispecific antibody recognizes and binds LOX-1, and the other arm recognizes and binds the “A” group of the labeled compound of the present invention. For example, a fusion polypeptide such as those disclosed in EP 1 046 652 A1 can be synthesized with the exception that a portion of the fusion polypeptide recognizes LOX-1 (instead of being LOX-1 as described therein) such as an antibody thereto. Bispecific antibodies can be prepared in accordance with U.S. Patent Application Publication No. 20020006379 to Hansen, et al., the disclosure of which is incorporated by reference herein in its entirety. Thus, a bispecific antibody can be prepared using the known techniques described above from known antibodies to LOX-1, or from antibodies raised using known techniques. One arm of the bispecific antibody will bind to and localize at the overexpression site of the LOX-1 protein. A labeled compound then can be synthesized using the guidelines provided herein whereby the “A” portion of the ligand binds to the free arm of the bispecific antibody, instead of the LOX-1 receptor itself. Imaging then can be carried out to detect the presence and concentration of LOX-1, whereby the imaging can be conducted on a routine basis (e.g., every week, month, year, etc.) to detect any overexpression. The imaging method therefore is capable of detecting the onset of cardiovascular disease.

The invention also encompasses a composition comprising the labeled compounds, as well as a kit for imaging a targeted tissue. The kit preferably comprises a composition comprising the labeled compound of the invention, or optionally, comprises two compositions; one containing an F-18 precursor, and the other containing the remaining portion of the compound (e.g., (L)ₙ-A). These two compositions can be mixed just prior to administration to the subject, thereby preserving the life of the F-18 radionuclide.

The invention now will be explained with reference to the following non-limiting examples.

### EXAMPLES

### Example 1

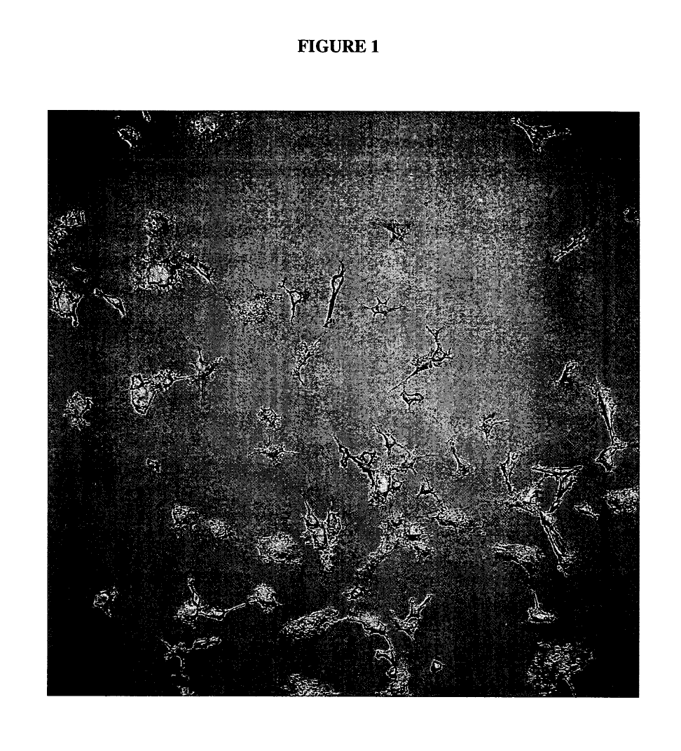

A peptide was conjugated with fluorescein (Fl-KKGG-FQTPPQL) and was shown to bind to human endothelial coronary artery cells (HCAECs) which are known in the literature to express LOX-1. The amino acid sequence of human LOX-1 is shown in FIG. 2. An image of HCAECs grown in glass slides treated with this peptide obtained using a fluorescent confocal microsope is shown in FIG. 1; the fluorescent image (shows fluorescently tagged peptide as bright green) is overlaid with the transmitted light image (shows outline of cells). The example reveals that the peptide above was localized on the cells. The experimental conditions for imaging the peptide-labeled HCAECs are described previously.

### Example 2

A solution of polyclonal antibody (IgG) was produced by Invitrogen Corporation, (Carlsbad, Calif.) against the sequence Arg-Gly-Ala-Val-Tyr-Ala-Glu-Asn-Cys-Ile at a concentration of 1.5 mg/mL. Three aliquots containing 250 μg (166 μL) each were transferred to 1.5 mL Eppendorf tubes and maintained at 0° C. The solutions were treated with NaHCO₃ (1M, 20 μL) and gently inverted. In a separate tube, a solution of 5-carboxyfluorescein-N-hydroxysuccinate ester in DMF (1 mg/mL) was prepared. The antibody solutions were treated with 5, 20 or 50 equivalents of the fluorescein/DMF solutions (3.95, 15.8 and 39 μL respectively). The highest concentration of DMF was 17%. The tubes were allowed to warm to room temperature over 1 hour and gently inverted every 15 minutes to assure mixing. During this time PD-10 columns were equilibrated with PBS and eluted until the sorbent bed was exposed.

The entire reaction mixtures were transferred to the columns and eluted with PBS. The fast moving yellow band was clearly visible and was collected in glass scintillation tubes (approximate eluted volume 3 mL). The purified labeled antibody samples were then evaluated using a Dot Blot technique against the LOX-1 antigen (Arg-Gly-Ala-Val-Tyr-Ala-Glu-Asn-Cys-Ile).

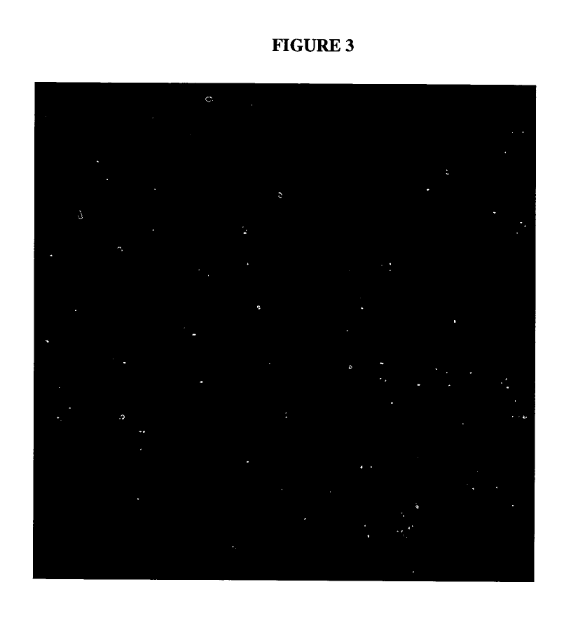

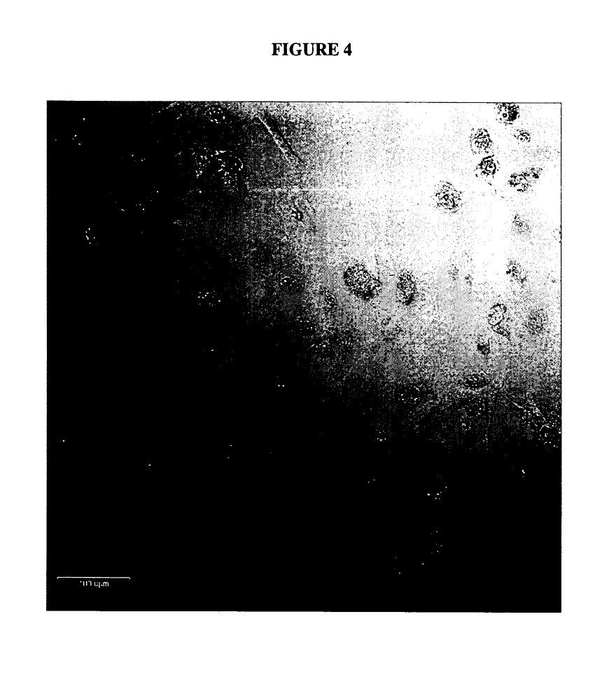

An image of HCAECs treated with this fluorescein-labeled polyclonal antibody is shown in FIGS. 3 and 4. The image was obtained using a laser confocal microscope as follows: HCAE cells were laid on and grown in wells on a standard commercial 96-well plate (Becton-Dickenson, Franklin Lakes, N.J.). Then, about 10 μL of 1 mg/ml labeled antibody aqueous solution was added to the cells and incubated for 1 hour. Subsequently, the cells were washed with PBS buffer three times. After a final wash with buffer, the cells were imaged. Images were acquired using an OLYMPUS laser scanning confocal microscope, model Fluoview 300, using Ar-ion laser (selecting 488 nm line) and a 510-nm long-pass filter. Images were acquired using two channels: reflected light and fluorescent mode channel. FIG. 3 is the fluorescent image (shows fluorescently tagged antibody as bright green), while FIG. 4 is the transmitted light image (shows the outline of cells). This example reveals that the antibody above was localized on the cells.

The results of this experiment confirmed that antibody immunoreactivity was not compromised in any of the samples and that adequate incorporation of dye had been accomplished in all cases. Further characterization by PAGE gel analysis confirmed protein integrity and that only the 150 kDa band contained the fluorescent label. Whole cell binding of the labeled antibody was then observed using the previously described binding protocol with HCAE cells.

### Example 3

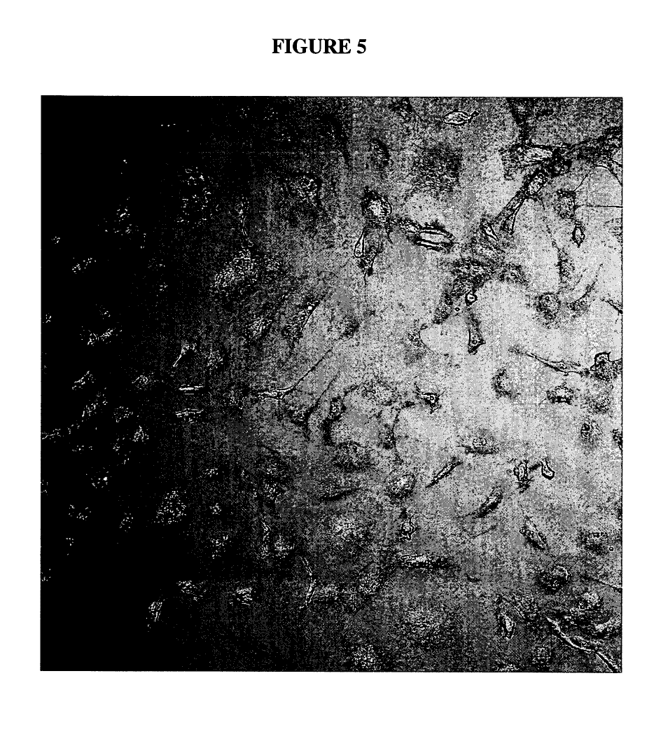

A sequence-scrambled version of the peptide described in Example 1 above was synthesized and conjugated with fluorescein in a similar manner as that described above. The scrambled sequence bound to fluorescein was Fl-KGKG-QPLFTPQ. This comparative peptide was contacted with HCAEC's and imaged in the same manner as described in Example 1 above. The results are shown in FIG. 5. As can be seen from FIG. 5, the sequence-scrambled version of the peptide had a weaker binding affinity for the HCAEC cells.

The invention has been described with reference to particularly preferred embodiments. Those skilled in the art will appreciate that various modifications may be made to the invention without departing from the spirit and scope thereof.

1. A compound having the formula S-(L)ₙ-A, wherein S is a signal providing structural unit that provides a signal that can be detected in vivo or detected in vitro, L links S to A, A is a peptide that binds to LOX-1, and n is either 0 or 1.

2. The compound of claim 1, wherein S is selected from the group consisting of a luminescent dye, a radionuclide, a near infrared dye, a magnetically active isotope, a superparamagnetic particle, a metal ion having a Z value of greater than 50, an encapsulated species, and a combination thereof.

3. The compound of claim 1, wherein S is selected from the group consisting of fluorescein, ¹¹C, ¹⁸F, ⁵²Fe, ⁶²Cu, ⁶⁴Cu, ⁶⁷Cu, ⁶⁷Ga, ⁶⁸Ga, ⁸⁶Y, ⁸⁹Zr, ^{94m}Tc, ⁹⁴Tc, ^{99m}Tc, ¹¹¹In, ¹²³I, ¹²⁴I, ¹²⁵I, ¹³¹I, ¹⁵⁴⁻¹⁵⁸Gd, ¹⁷⁵Lu, superparamagnetic iron oxide nanoparticles, heavy metal ions, gas-filled microbubbles, optical dyes, porphyrins, texaphyrins, highly iodinated organic compounds chelates thereof, polymers containing at least one of the aforementioned components, endohedral fullerenes containing at least one of the aforementioned, and mixtures thereof.

4. The compound of claim 2, wherein S is a luminescent dye.

5. The compound of claim 4, wherein the luminescent dye is fluorescein, rhodamine, Alexa, or derivatives thereof.

6. The compound of claim 2, wherein S is a radionuclide.

7. The compound of claim 6, wherein the radionuclide is a positron emitter.

8. The compound of claim 7, wherein the positron emitter is selected from ¹⁸F and ¹¹C.

9. The compound of claim 6, wherein the radionuclide is a gamma emitter.

10. The compound of claim 2, wherein S is selected from the group consisting of a near infrared dye, an infrared dye, Cye dyes, and mixtures thereof.

11. The compound of claim 2, wherein S is a magnetically active isotope.

12. The compound of claim 11, wherein the magnetically active isotope is paramagnetic.

13. The compound of claim 12, wherein the magnetically active isotope is an isotope of gadolinium.

14. The compound of claim 2, wherein S is a superparamagnetic particle.

15. The compound of claim 14, wherein the superparamagnetic particle is a nanoparticle.

16. The compound according to claim 15, wherein the nanoparticle comprises at least one of iron oxide and elemental iron.

17. The compound according to claim 2, wherein S is an element having a Z value of greater than about 50.

18. The compound according to claim 17, wherein the element having a Z value of greater than about 50 is iodine or bismuth.

19. The compound according to claim 2, wherein S is an encapsulated species.

20. The compound according to claim 19, wherein the encapsulated species is selected from the group consisting of a micelle, a liposome, a polysome, and a gas-filled microbubble.

21. The compound according to claim 1, wherein L is an organic radical having a valence of at least 2.

22. The compound according to claim 21, wherein the organic radical is covalently bound to both group S and group A.

23. The compound according to claim 21, wherein the organic radical is ionically bound to one of group S and group A.

24. The compound according to claim 23, wherein the organic radical is ionically bound to both group S and group A.

25. The compound according to claim 21, wherein the organic radical comprises between 1 and about 10,000 carbon atoms.

26. The compound according to claim 25, wherein the organic radical is selected from the group consisting of alkylene, arylene, cycloalkylene, aminoalklylene, aminoarylene, aminocycloalkylene, thioalkylene, thioarylene, thiocycloalkylene, oxyalkylene, oxyarylene, oxycycloalkylene, acylalkylene, acylarylene, acylcycloalkylene units, and combinations thereof.

27. The compound of claim 26, wherein the acylarylene unit is a 4-acylphenylene group having the following structure:

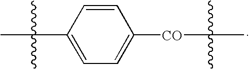

28. The compound of claim 21, wherein the organic radical is a metal chelating agent.

29. The compound according to claim 28, wherein the metal chelating agent binds at least one metal cation selected from the group consisting of cations of ⁵²Fe, ⁶²Cu, 64Cu, ⁶⁷Cu, ⁶⁷Ga, ⁶⁸Ga, ⁸⁶Y, ⁸⁹Zr, ^{94m}Tc, ⁹⁴Tc, ^{99m}Tc, ¹¹¹In, ¹⁵⁴⁻¹⁵⁸Gd, and ¹⁷⁵Lu.

30. The compound of claim 29, wherein the metal chelating agent is selected from the group consisting of DTPA, 1,4,7-triaza-cyclononane-N,N′,N″-triacetic acid (NOTA), p-bromoacetamido-benyl-tetraethylaminetetraacetic acid (TETA), 1,4,7,10-tetraazacyclododecanetetraacetic acid (DOTA), EDTA, CHXa.

31. The compound of claim 1, wherein n is 1, and L is selected from the group consisting of lysine-glycine analogs, lysine-glycine derivatives, lysine-glycine variants, cyclohexyl alanine, DTPA, 1,4,7-triaza-cyclononane-N,N′,N″-triacetic acid (NOTA), p-bromoacetamido-benyl-tetraethylaminetetraacetic acid (TETA), 1,4,7,10-tetraazacyclododecanetetraacetic acid (DOTA), and mixtures thereof.

32. The compound of claim 31, wherein L is KKGG (SEQ ID NO: 6).

33. The compound of claim 1, wherein A is selected from the group consisting of LSXPP (X=I, R) (SEQ ID NO: 1), TPP, LTPAXA (X=T, R) (SEQ ID NO: 2), MTTPPLT (SEQ ID NO: 3), LTRPX (X=Y, L) (SEQ ID NO: 4), MTAXPI (X=P, R) (SEQ ID NO: 5), MPQ, and mixtures, fragments, fusion peptides, derivatives, variants, and homologues thereof.

34. The compound of claim 33, wherein A is TPP or variants thereof.

35. A composition comprising a compound having the formula S-(L)ₙ-A, wherein S provides a signal that can be detected in vivo or detected in vitro, L links S to A, A is a peptide comprising an amino acid sequence that binds to LOX-1, and n is either 0 or 1.

36. The composition of claim 35, wherein S is selected from the group consisting of a luminescent dye, a radionuclide, a near infrared dye, a magnetically active isotope, a superparamagnetic particle, a metal ion having a Z value of greater than 50, an encapsulated species, and a combination thereof.

37. The composition of claim 35, wherein S is selected from the group consisting of fluorescein, ¹¹C, ¹⁸F, ⁵²Fe, ⁶²Cu, ⁶⁴Cu, ⁶⁷Cu, ⁶⁷Ga, ⁶⁸Ga, ⁸⁶Y, ⁸⁹Zr, ^{94m}Tc, ⁹⁴Tc, ^{99m}Tc, ¹¹¹In, ¹²³I, ¹²⁴I, ¹²⁵I, ¹³¹I, ¹⁵⁴⁻¹⁵⁸Gd, ¹⁷⁵Lu, superparamagnetic iron oxide nanoparticles, heavy metal ions, gas-filled microbubbles, optical dyes, porphyrins, texaphyrins, highly iodinated organic compounds chelates thereof, polymers containing at least one of the aforementioned components, endohedral fullerenes containing at least one of the aforementioned, and mixtures thereof.

38. The composition of claim 37, wherein S is selected from ¹⁸F and ¹¹C.

39. The composition of claim 35, wherein L is an organic radical selected from the group consisting of alkylene, arylene, cycloakylene, aminoaklylene, aminoarylene, aminocycloalkylene, thioalkylene, thioarylene, thiocycloalkylene, oxyalkylene, oxyarylene, oxycycloalkylene, acylalkylene, acylarylene, acylcycloalkylene units, and combinations thereof.

40. The composition of claim 39, wherein the acylarylene unit is a 4-acylphenylene group having the following structure:

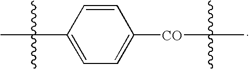

41. The composition of claim 39, wherein the organic radical is a metal chelating agent.

42. The composition according to claim 41, wherein the metal chelating agent binds at least one metal cation selected from the group consisting of cations of ⁵²Fe, ⁶²Cu, ⁶⁴Cu, ⁶⁷Cu, ⁶⁷Ga, ⁶⁸Ga, ⁸⁶Y, ⁸⁹Zr, ^{94m}Tc, ⁹⁴Tc, ^{99m}Tc, ¹¹¹In, ¹⁵⁴⁻¹⁵⁸Gd, and ¹⁷⁵Lu.

43. The composition of claim 42, wherein the metal chelating agent is selected from the group consisting of DTPA, 1,4,7-triaza-cyclononane-N,N′,N″-triacetic acid (NOTA), p-bromoacetamido-benyl-tetraethylaminetetraacetic acid (TETA), 1,4,7,10-tetraazacyclododecanetetraacetic acid (DOTA), EDTA, CHXa.

44. The composition of claim 35, wherein n is 1, and L is selected from the group consisting of lysine-glycine analogs, lysine-glycine derivatives, lysine-glycine variants, cyclohexyl alanine, DTPA, 1,4,7-triaza-cyclononane-N,N′,N″-triacetic acid (NOTA), p-bromoacetamido-benyl-tetraethylaminetetraacetic acid (TETA), 1,4,7,10-tetraazacyclododecanetetraacetic acid (DOTA), and mixtures thereof.

45. The composition of claim 44, wherein L is KKGG (SEQ ID NO: 6).

46. The compound of claim 35, wherein A is selected from the group consisting of LSXPP (X=I, R) (SEQ ID NO: 1), TPP, LTPAXA (X=T, R) (SEQ ID NO: 2), MTTPPLT (SEQ ID NO: 3), LTRPX (X=Y, L) (SEQ ID NO: 4), MTAXPI (X=P, R) (SEQ ID NO: 5), MPQ, and mixtures, fragments, fusion peptides, derivatives, variants, and homologues thereof.

47. The composition of claim 46, wherein A is TPP or variants thereof.

48. A kit comprising the composition of claim 8.

49. A method of imaging a tissue to detect the presence and/or amount of LOX-1, comprising:
administering to a mammal the compound of claim 1;
optionally administering a clearing agent to remove any compound that is not bound to LOX-1; and
subjecting the mammal to imaging effective to detect the signal generated by S to thereby detect the presence and/or amount of LOX-1.

50. The method of claim 49, wherein the mammal is suspected of a disease or disorder related to the expression of LOX-1.

51. The method of claim 50, wherein the disease or disorder is selected from the group consisting of atherosclerosis, vulnerable plaque, coronary artery disease, renal disease, thrombosis, transient ischemia due to clotting, stroke, myocardial infarction, organ transplant, organ failure, and hypercholesterolemia.

52. The method of claim 49, wherein the imaging effective to detect S is positron emission tomography.

53. The method of claim 52, wherein S is selected from ¹⁸F and ¹¹C.

54. The method of claim 49, wherein S is selected from the group consisting of a luminescent dye, a radionuclide, a near infrared dye, a magnetically active isotope, a superparamagnetic particle, a metal ion having a Z value of greater than 50, an encapsulated species, and a combination thereof.

55. The method of claim 49, wherein S is selected from the group consisting of fluorescein, ¹¹C, ¹⁸F, ⁵²Fe, ⁶²Cu, ⁶⁴Cu, ⁶⁷Cu, ⁶⁷Ga, ⁶⁸Ga, ⁸⁶Y, ⁸⁹Zr, ^{94m}Tc, ⁹⁴Tc, ^{99m}Tc, ¹¹¹In, ¹²³I, ¹²⁴I, ¹²⁵I, ¹³¹I, ¹⁵⁴⁻¹⁵⁸Gd, ¹⁷⁵Lu, superparamagnetic iron oxide nanoparticles, heavy metal ions, gas-filled microbubbles, optical dyes, porphyrins, texaphyrins, highly iodinated organic compounds chelates thereof, polymers containing at least one of the aforementioned components, endohedral fullerenes containing at least one of the aforementioned, and mixtures thereof.

56. The method of claim 49, wherein L is an organic radical selected from the group consisting of alkylene, arylene, cycloakylene, aminoaklylene, aminoarylene, aminocycloalkylene, thioalkylene, thioarylene, thiocycloalkylene, oxyalkylene, oxyarylene, oxycycloalkylene, acylalkylene, acylarylene, acylcycloalkylene units, and combinations thereof.

57. The method of claim 55, wherein the acylarylene unit is a 4-acylphenylene group having the following structure:

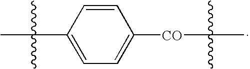

58. The method of claim 55, wherein the organic radical is a metal chelating agent.

59. The method according to claim 58, wherein the metal chelating agent binds at least one metal cation selected from the group consisting of cations of ⁵²Fe, ⁶²Cu, ⁶⁴Cu, ⁶⁷Cu, ⁶⁷Ga, ⁶⁸Ga, ⁸⁶Y, ⁸⁹Zr, ^{94m}Tc, ⁹⁴Tc, ^{99m}Tc, ¹¹¹In, ¹⁵⁴⁻¹⁵⁸Gd, and ¹⁷⁵Lu.

60. The method of claim 59, wherein the metal chelating agent is selected from the group consisting of DTPA, 1,4,7-triaza-cyclononane-N,N′,N″-triacetic acid (NOTA), p-bromoacetamido-benyl-tetraethylaminetetraacetic acid (TETA), 1,4,7,10-tetraazacyclododecanetetraacetic acid (DOTA), EDTA, CHXa.

61. The method of claim 49, wherein n is 1, and L is selected from the group consisting of lysine-glycine analogs, lysine-glycine derivatives, lysine-glycine variants, cyclohexyl alanine, DTPA, 1,4,7-triaza-cyclononane-N,N′,N″-triacetic acid (NOTA), p-bromoacetamido-benyl-tetraethylaminetetraacetic acid (TETA), 1,4,7,10-tetraazacyclododecanetetraacetic acid (DOTA), and mixtures thereof.

62. The method of claim 61, wherein L is KKGG (SEQ ID NO: 6).

63. The method of claim 49, wherein A is selected from the group consisting of LSXPP (X=I, R) (SEQ ID NO: 1), TPP, LTPAXA (X=T, R) (SEQ ID NO: 2), MTTPPLT (SEQ ID NO: 3), LTRPX (X=Y, L) (SEQ ID NO: 4), MTAXPI (X=P, R) (SEQ ID NO: 5), MPQ, and mixtures, fragments, fusion peptides, derivatives, variants, and homologues thereof.

64. The method of claim 63, wherein A is TPP or variants thereof.

65. A method of monitoring the efficacy of therapies for treating atherosclerosis comprising:
administering to a mammal the compound of claim 1;
optionally administering a clearing agent to remove the compound that is not bound to LOX-1;
subjecting the mammal to imaging effective to detect the signal generated by S to thereby detect the amount of LOX-1; and
repeating the administration and imaging procedures at least once over a period of time to detect the difference in amount of LOX-1.

66. The method of claim 65, wherein the mammal is suspected of a disease or disorder caused by expression of LOX-1.

67. The method of claim 65 wherein the imaging effective to detect S is positron emission tomography.

68. The method of claim 67, wherein S is selected from ¹⁸F and ¹¹C.

69. The method of claim 65, wherein S is selected from the group consisting of a luminescent dye, a radionuclide, a near infrared dye, a magnetically active isotope, a superparamagnetic particle, a metal ion having a Z value of greater than 50, an encapsulated species, and a combination thereof.

70. The method of claim 65, wherein S is selected from the group consisting of fluorescein, ¹¹C, ¹⁸F, ⁵²Fe, ⁶²Cu, ⁶⁴Cu, ⁶⁷Cu, ⁶⁷Ga, ⁶⁸Ga, ⁸⁶Y, ⁸⁹Zr, ^{94m}Tc, ⁹⁴Tc, ^{99m}Tc, ¹¹¹In, ¹²³I, ¹²⁴I, ¹²⁵I, ¹³¹I, ¹⁵⁴⁻¹⁵⁸Gd, ¹⁷⁵Lu, superparamagnetic iron oxide nanoparticles, heavy metal ions, gas-filled microbubbles, optical dyes, porphyrins, texaphyrins, highly iodinated organic compounds chelates thereof, polymers containing at least one of the aforementioned components, endohedral fullerenes containing at least one of the aforementioned, and mixtures thereof.

71. The method of claim 65, wherein L is an organic radical selected from the group consisting of alkylene, arylene, cycloakylene, aminoaklylene, aminoarylene, aminocycloalkylene, thioalkylene, thioarylene, thiocycloalkylene, oxyalkylene, oxyarylene, oxycycloalkylene, acylalkylene, acylarylene, acylcycloalkylene units, and combinations thereof.

72. The method of claim 71, wherein the acylarylene unit is a 4-acylphenylene group having the following structure:

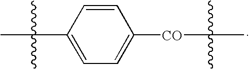

73. The method of claim 71, wherein the organic radical is a metal chelating agent.

74. The method according to claim 73, wherein the metal chelating agent binds at least one metal cation selected from the group consisting of cations of ⁵²Fe, ⁶²Cu, ⁶⁴Cu, ⁶⁷Cu, ⁶⁷Ga, ⁶⁸Ga, ⁸⁶Y, ⁸⁹Zr, ^{94m}Tc, 94Tc, ^{99m}Tc, ¹¹¹In, ¹⁵⁴⁻¹⁵⁸Gd, and ¹⁷⁵Lu.

75. The method of claim 74, wherein the metal chelating agent is selected from the group consisting of DTPA, 1,4,7-triaza-cyclononane-N,N′,N″-triacetic acid (NOTA), p-bromoacetamido-benyl-tetraethylaminetetraacetic acid (TETA), 1,4,7,10-tetraazacyclododecanetetraacetic acid (DOTA), EDTA, CHXa.

76. The method of claim 65, wherein n is 1, and L is selected from the group consisting of lysine-glycine analogs, lysine-glycine derivatives, lysine-glycine variants, cyclohexyl alanine, DTPA, 1,4,7-triaza-cyclononane-N,N′,N″-triacetic acid (NOTA), p-bromoacetamido-benyl-tetraethylaminetetraacetic acid (TETA), 1,4,7,10-tetraazacyclododecanetetraacetic acid (DOTA), and mixtures thereof.

77. The method of claim 76, wherein L is KKGG (SEQ ID NO: 6).

78. The method of claim 65, wherein A is selected from the group consisting of LSXPP (X=I, R) (SEQ ID NO: 1), TPP, LTPAXA (X=T, R) (SEQ ID NO: 2), MTTPPLT (SEQ ID NO: 3), LTRPX (X=Y, L) (SEQ ID NO: 4), MTAXPI (X=P, R) (SEQ ID NO: 5), MPQ, and mixtures, fragments, fusion peptides, derivatives, variants, and homologues thereof.

79. The method of claim 78, wherein A is TPP or variants thereof.

80. The method of claim 66, wherein the disease or disorder is selected from the group consisting of atherosclerosis, vulnerable plaque, coronary artery disease, renal disease, thrombosis, transient ischemia due to clotting, stroke, myocardial infarction, organ transplant, organ failure, and hypercholesterolemia.
